(19) United States
(12) Patent Application Publication
(10) Pub. No.: US 2025/0114034 A1
(43) Pub. Date: Apr. 10, 2025

(54) Time Before Sound Sleep Metric Facilitating Sleep Quality Assessment and Alteration

(71) Applicant: Google LLC, Mountain View, CA (US)

(72) Inventors: Alicia Yolanda Kokoszka, San Francisco, CA (US); Karla Theresa Gleichauf, San Francisco, CA (US)

(21) Appl. No.: 18/865,644

(22) PCT Filed: Jun. 30, 2022
(86) PCT No.: PCT/US2022/035750

§ 371 Date: Nov. 13, 2024

(51) Int. Cl. A61B 5/00

(52) U.S. Cl. CPC A61B 5/4812; A61B 5/4815; A61B 5/74

(57) ABSTRACT

According to an embodiment, a computing device can include one or more processors and one or more computer-readable media that store instructions that, when executed by the processor(s), cause the computing device to perform operations. The operations can include obtaining a plurality of sleep stages associated with a sleep session of a user. The sleep session can be at least partially defined by an estimated bedtime of the user. The operations can further include identifying, in the plurality of sleep stages, one or more defined sleep stages indicative of a defined sleep state of the user. The operations can further include calculating a time before sound sleep (TBSS) metric based at least in part on the estimated bedtime of the user and a start time of the defined sleep stage(s). The operations can further include performing one or more operations based at least in part on the TBSS metric.

### FIELD

The present disclosure relates generally to sleep quality assessment and alteration. More particularly, the present disclosure relates to calculating a time before sound sleep metric and using the time before sound sleep metric to facilitate sleep quality assessment and alteration.

### BACKGROUND

Assessing how long it takes a user to fall asleep is difficult for both traditional sleep detection technology and wearable devices such as, for example, wrist-worn physiological monitoring devices. Some existing wrist-worn physiological monitoring devices use accelerometer data to determine an estimated bedtime of a user (e.g., a time at which the user enters a restful pre-sleep state) and/or a combination of heart rate data and accelerometer data to determine the sleep stages of the user.

A problem with such wrist-worn physiological monitoring devices described above is that challenges exist in attempting to accurately and consistently distinguish between when a user is in a relaxing state of stillness (e.g., watching television in bed, reading) and when the user is in a true sleep state. That is, for example, such wrist-worn physiological monitoring devices may not accurately and consistently distinguish between the estimated bedtime of the user and the time at which the user enters the sound sleep state. As such, another problem with such wrist-worn physiological monitoring devices is that they often do not reflect a user's perception of their sleep onset latency (i.e., the time it takes the user to fall asleep after their estimated bedtime).

### SUMMARY

Aspects and advantages of embodiments of the present disclosure will be set forth in part in the following description, or can be learned from the description, or can be learned through practice of the embodiments.

According to one example embodiment, a computing device can include one or more processors and one or more computer-readable media that store instructions that, when executed by the one or more processors, cause the computing device to perform operations. The operations can include obtaining a plurality of sleep stages associated with a sleep session of a user. The sleep session can be at least partially defined by an estimated bedtime of the user. The operations can further include identifying, in the plurality of sleep stages, one or more defined sleep stages indicative of a defined sleep state of the user. The operations can further include calculating a time before sound sleep metric based at least in part on the estimated bedtime of the user and a start time of the one or more defined sleep stages. The operations can further include performing one or more operations based at least in part on the time before sound sleep metric.

According to another example embodiment, a computer-implemented method of assessing sleep quality and facilitating sleep quality alteration can include obtaining, by a computing device operatively coupled to one or more processors, a plurality of sleep stages associated with a sleep session of a user. The sleep session can be at least partially defined by an estimated bedtime of the user. The computer-implemented method can further include identifying, by the computing device, in the plurality of sleep stages, one or more defined sleep stages indicative of a defined sleep state of the user. The computer-implemented method can further include calculating, by the computing device, a time before sound sleep metric based at least in part on the estimated bedtime of the user and a start time of the one or more defined sleep stages. The computer-implemented method can further include performing, by the computing device, one or more operations based at least in part on the time before sound sleep metric.

According to another example embodiment, one or more computer-readable media that can store instructions that, when executed by one or more processors of a computing device, can cause the computing device to perform operations. The operations can include obtaining a plurality of sleep stages associated with a sleep session of a user. The sleep session can be at least partially defined by an estimated bedtime of the user. The operations can further include identifying, in the plurality of sleep stages, one or more defined sleep stages indicative of a defined sleep state of the user. The operations can further include calculating a time before sound sleep metric based at least in part on the estimated bedtime of the user and a start time of the one or more defined sleep stages. The operations can further include performing one or more operations based at least in part on the time before sound sleep metric.

These and other features, aspects, and advantages of various embodiments of the present disclosure will become better understood with reference to the following description and appended claims. The accompanying drawings, which are incorporated in and constitute a part of this specification, illustrate example embodiments of the present disclosure and, together with the description, serve to explain the related principles.

### BRIEF DESCRIPTION OF THE DRAWINGS

Detailed discussion of embodiments directed to one of ordinary skill in the art is set forth in the specification, which makes reference to the appended figures, in which:

FIGS. 1, 2, and 3 each illustrate a perspective view of an example, non-limiting wearable device according to one or more example embodiments of the present disclosure.

FIG. 4 illustrates a block diagram of an example, non-limiting device according to one or more example embodiments of the present disclosure.

FIGS. 5 and 6 each illustrate a diagram of an example, non-limiting sleep quality management system according to one or more example embodiments of the present disclosure.

FIGS. 7A, 7B, and 7C each illustrate a diagram of example, non-limiting sleep stages according to one or more example embodiments of the present disclosure.

FIG. 8 illustrates a flow diagram of an example, non-limiting computer-implemented method according to one or more example embodiments of the present disclosure.

Repeated use of reference characters and/or numerals in the present specification and/or figures is intended to represent the same or analogous features, elements, or operations of the present disclosure. Repeated description of reference characters and/or numerals that are repeated in the present specification is omitted for brevity.

### DETAILED DESCRIPTION

### Overview

As referred to herein, the terms “includes” and “including” are intended to be inclusive in a manner similar to the term “comprising.” As referenced herein, the terms “or” and “and/or” are generally intended to be inclusive, that is (i.e.), “A or B” or “A and/or B” are each intended to mean “A or B or both.” As referred to herein, the terms “first,” “second,” “third,” and so on, can be used interchangeably to distinguish one component or entity from another and are not intended to signify location, functionality, or importance of the individual components or entities. As referenced herein, the terms “couple,” “couples,” “coupled,” and/or “coupling” refer to chemical coupling (e.g., chemical bonding), communicative coupling, electrical and/or electromagnetic coupling (e.g., capacitive coupling, inductive coupling, direct and/or connected coupling, etc.), mechanical coupling, operative coupling, optical coupling, and/or physical coupling.

As referenced herein, the term “system” can refer to hardware (e.g., application specific hardware), computer logic that executes on a general-purpose processor (e.g., a central processing unit (CPU)), and/or some combination thereof. In some embodiments, a “system” described herein can be implemented in hardware, application specific circuits, firmware, and/or software controlling a general-purpose processor. In some embodiments, a “system” described herein can be implemented as program code files stored on a storage device, loaded into a memory and executed by a processor, and/or can be provided from computer program products, for example, computer-executable instructions that are stored in a tangible computer-readable storage medium (e.g., random-access memory (RAM), hard disk, optical media, magnetic media).

Example aspects of the present disclosure are directed to assessing sleep quality of a user and facilitating alteration (e.g., improvement) of the user's sleep quality based on such assessment. More specifically, example embodiments described herein are directed to calculating a time before sound sleep (TBSS) metric associated with a user and further using the TBSS metric to perform one or more operations that can facilitate alteration (e.g., improvement) of the user's sleep quality.

As referenced herein, the “time before sound sleep (TBSS) metric” or “TBSS metric” is defined as a time period between an estimated bedtime of a user and a time when the user enters a sound sleep state. As used herein, the term “estimated bedtime” of a user is defined as an estimated time at which the user enters a restful pre-sleep state (e.g., a relaxing state of stillness). As referred to herein, the term “sound sleep state” is defined as a continuous and/or quality sleep state (e.g., a deep sleep, a rapid eye movement (REM) sleep, and/or a relatively high-quality sleep state where the user is not moving (e.g., not tossing and turning)). For example, as referenced herein, the term “sound sleep state” is defined as any one of: 1) a first instance of deep sleep; 2) a first instance of REM sleep; or 3) a first instance of light sleep in a light sleep stage bout of, for example, 20) minutes or longer that is not broken up by other sleep stages (e.g., not interrupted by a deep sleep stage, an REM sleep stage, or a wake stage).

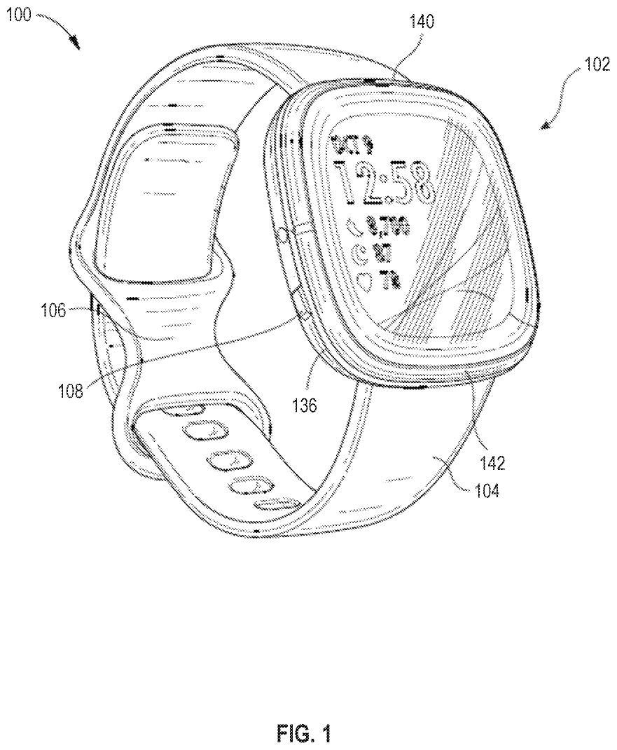

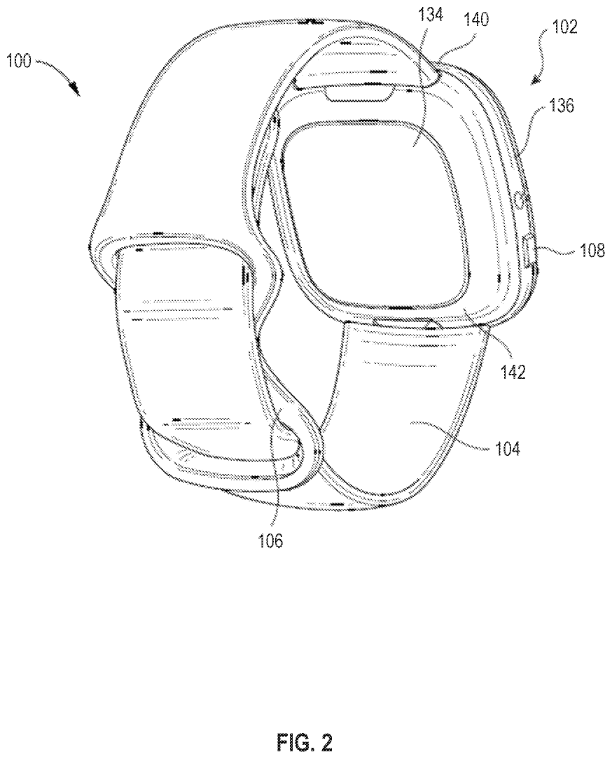

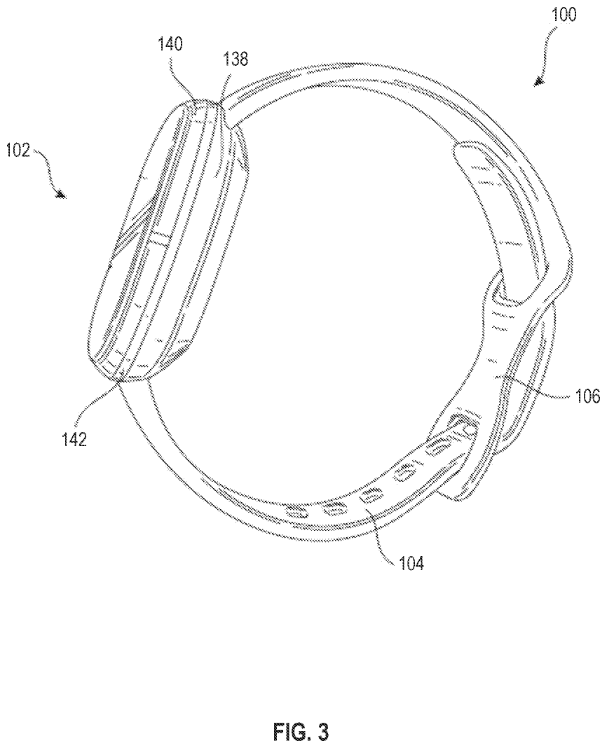

According to one or more example embodiments of the present disclosure, a computing device such as, for instance, wearable device 100 described below with reference to the example embodiments depicted in FIGS. 1, 2, 3, and 4, can assess sleep quality of a user and facilitate alteration (e.g., improvement) of the user's sleep quality based on such assessment. More specifically, in at least one embodiment described herein, such a computing device can calculate a TBSS metric associated with a user and further use such a TBSS metric to perform one or more operations that can facilitate alteration (e.g., improvement) of the user's sleep quality. In some embodiments, the computing device can constitute and/or include, for instance, a physiological monitoring device, a wearable computing device, a wearable physiological monitoring device (e.g., a wrist-worn device, a chest strap device), and/or another computing device that can calculate a TBSS metric associated with a user and further use such a TBSS metric to perform one or more operations that can facilitate alteration (e.g., improvement) of the user's sleep quality.

In at least one embodiment of the present disclosure, to assess sleep quality of a user and facilitate alteration (e.g., improvement) of the user's sleep quality based on such assessment, the above-described computing device (e.g., wearable device 100) can perform operations that can include, but are not limited to: obtaining a plurality of sleep stages associated with a sleep session of a user, the sleep session at least partially defined by an estimated bedtime of the user; identifying, in the plurality of sleep stages, one or more defined sleep stages indicative of a defined sleep state of the user; calculating a time before sound sleep (TBSS) metric based at least in part on the estimated bedtime of the user and a start time of the one or more defined sleep stages; and performing one or more operations based at least in part on the TBSS metric. In this or another embodiment, the TBSS metric can be indicative of a time period between the estimated bedtime of the user and a time when the user enters the defined sleep state. In this or another embodiment, the estimated bedtime of the user can constitute the above-defined estimated bedtime and the defined sleep state can constitute the above-defined sound sleep state.

In one or more embodiments described herein, the plurality of sleep stages that can be associated with a sleep session of a user (e.g., a sleep session lasting one or more hours) can include, for example, a wake stage, a light sleep stage, a deep sleep stage, an REM sleep stage, and/or another sleep stage. For example, in one embodiment, the plurality of sleep stages can include: a wake stage where the user is awake; a light sleep stage where the user is in a restful pre-sleep state and/or a relaxing state of stillness; a deep sleep stage where the user is in a true, continuous, and/or quality sleep state; an REM sleep stage where the user experiences REM and is thus in a true, continuous, and/or quality sleep state; and/or another sleep stage.

In at least one embodiment of the present disclosure, the one or more defined sleep stages that can be indicative of the above-described defined sleep state of the user (e.g., the sound sleep state defined above) can constitute and/or include a defined quantity of one or more of the sleep stages in the plurality of sleep stages described above. For example, in one embodiment, the one or more defined sleep stages can constitute and/or include: a defined quantity (e.g., 10, 20, 30) of the light sleep stage; a defined quantity (e.g., 1, 2) of the deep sleep stage; and/or a defined quantity (e.g., 1, 2) of the REM sleep stage.

In some embodiments described herein, each sleep stage of the plurality of sleep stages and each defined sleep stage of the one or more defined sleep stages can be defined by a certain time interval (e.g., 30 seconds, 1 minute, 2 minutes) such that all sleep stages are defined by the same duration of time (e.g., 30 seconds, 1 minute, 2 minutes). For example, in one embodiment, each sleep stage of the plurality of sleep stages and each defined sleep stage of the one or more defined sleep stages can be 1 minute in duration. In some embodiments, each sleep stage of the plurality of sleep stages and each defined sleep stage of the one or more defined sleep stages can be defined by a certain time interval (e.g., 30 seconds, 1 minute, 2 minutes) that corresponds to a discrete time interval (e.g., 30 seconds, 1 minute, 2 minutes) in the user's sleep session.

In one or more embodiments of the present disclosure, a time at which a certain light sleep stage (e.g., the 1^{st} light sleep stage) begins in the user's sleep session can correspond to the estimated bedtime of the user as defined above. In some embodiments, a time at which the above-described one or more defined sleep stages begin can correspond to a time at which the user enters the defined sleep state (e.g., the sound sleep state defined above).

In one embodiment of the present disclosure, the one or more defined sleep stages described above can constitute and/or include a defined quantity of the light sleep stages such as, for instance, 20 light sleep stages (e.g., 20 consecutive light sleep stages). In this or another embodiment, the one or more defined sleep stages described above can constitute and/or include 20 consecutive, uninterrupted light sleep stages in the user's sleep session. As such, in this or another embodiment, the one or more defined sleep stages can constitute and/or include a 20-minute, uninterrupted bout of light sleep. In this or another embodiment, a time at which the 1^{st} light sleep stage begins in the user's sleep session can correspond to the estimated bedtime of the user as defined above. In this or another embodiment, a time at which the 1^{st} light sleep stage begins in the bout of 20 consecutive, uninterrupted light sleep stages can correspond to the time at which the user enters the sound sleep state defined above. That is, for instance, in this or another embodiment, a start time of the 1^{st} light sleep stage in the bout of 20 consecutive, uninterrupted light sleep stages can correspond to the time at which the user enters the sound sleep state defined above.

In another embodiment of the present disclosure, the one or more defined sleep stages described above can constitute and/or include a defined quantity of the deep sleep stages such as, for instance, I deep sleep stage. In this or another embodiment, a time at which the 1^{st} light sleep stage begins in the user's sleep session can correspond to the estimated bedtime of the user as defined above. In this or another embodiment, a time at which the deep sleep stage (e.g., the 1^{st} deep sleep stage) begins in the user's sleep session can correspond to the time at which the user enters the sound sleep state defined above.

In another embodiment of the present disclosure, the one or more defined sleep stages described above can constitute and/or include a defined quantity of the REM sleep stages such as, for instance, 1 REM sleep stage. In this or another embodiment, a time at which the 1^{st} light sleep stage begins in the user's sleep session can correspond to the estimated bedtime of the user as defined above. In this or another embodiment, a time at which the REM sleep stage (e.g., the 1^{st} REM sleep stage) begins in the user's sleep session can correspond to the time at which the user enters the sound sleep state defined above.

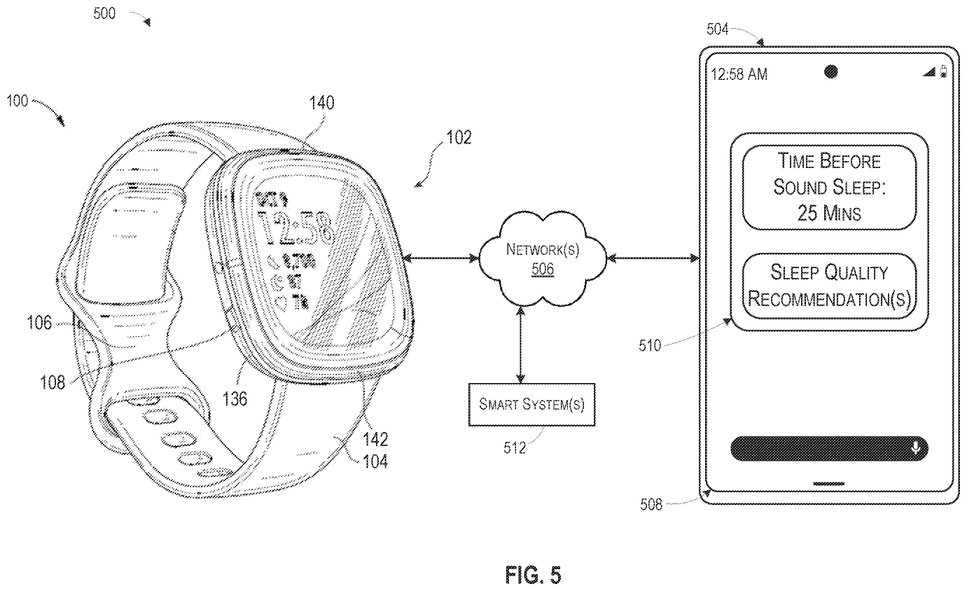

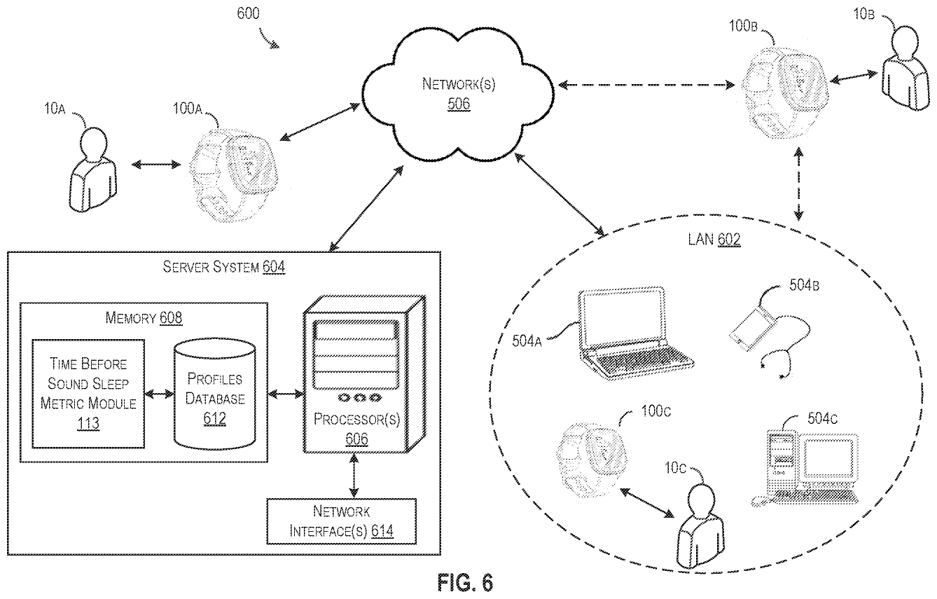

In one embodiment of the present disclosure, the above-described computing device (e.g., wearable device 100) can obtain the above-described plurality of sleep stages from another computing device that can generate such sleep stages using physiological data (e.g., accelerometer data, heart rate data, pulse-related data, respiratory-related data) corresponding to the user that was captured and/or collected (e.g., via an accelerometer, a photoplethysmography (PPG) sensor) during the user's sleep session. For example, in at least one embodiment, the computing device (e.g., wearable device 100) can obtain the plurality of sleep stages from external computing device 504, 504a, 504b, and/or 504c and/or server system 604 described below with reference to the example embodiments depicted in FIGS. 5 and 6. In another embodiment, the computing device (e.g., wearable device 100) can generate the plurality of sleep stages (e.g., as described below) using physiological data corresponding to the user that can be captured and/or collected by the computing device during the user's sleep session.

In some embodiments described herein, the computing device (e.g., wearable device 100) can include and/or be communicatively coupled to one or more physiological sensors such as, for example, an accelerometer and/or a PPG sensor that can respectively capture accelerometer data (e.g., motion data) and/or heart rate data (e.g., pulse-related data) corresponding to a user. In these or another embodiment, the computing device can operate the accelerometer and/or PPG sensor during a sleep session of a user to capture accelerometer data (e.g., motion data) and/or heart rate data (e.g., pulse-related data) corresponding to the user. In these or another embodiment, to generate the above-described plurality of sleep stages, the computing device can implement one or more classification modules, processes, techniques, algorithms, and/or models (e.g., machine learning models) that can classify and/or otherwise label periods of time in the user's sleep session (e.g., periods of 30 seconds, 1 minute, 2 minutes) with a certain sleep stage (e.g., wake, light, deep, REM) based on one or more features derived from the user's accelerometer data (e.g., motion data) and/or heart rate data (e.g., pulse-related data). In these or another embodiment, the computing device can generate the plurality of sleep stages using a classifier (e.g., a machine learning algorithm and/or model) such as, for example, a nearest neighbor classifier, a random forest classifier, a support vector machine, a decision tree, a neural network, a linear discriminant classifier, and/or another classifier.

In at least one embodiment of the present disclosure, the above-described classifier(s) that can be used by the computing device (e.g., wearable device 100) to generate the sleep stages can be trained on a known set of annotated sleep logs that can be produced from a plurality of sleep sessions of different users. For example, in this or another embodiment, such classifier(s) can be trained using features that can be extracted from accelerometer data (e.g., motion data) and/or heart rate (e.g., pulse-related data) that can be collected during sleep sessions of different users. In this or another embodiment, each sleep session of the different users can be conducted with the oversight of a professional and/or a trained sleep scorer using specialized equipment and/or in-depth analysis of each time interval being evaluated in each sleep session. As such, in this or another embodiment, the features that can be extracted from such accelerometer and/or heart rate data of the different users that has been collected during time periods that have been determined by a sleep scorer to be deep sleep stages can be used to train the above-described classifier to assign a deep sleep stage classification to certain time periods in the user's sleep session that have features that satisfy (e.g., match) the criteria of the classifier developed during such training.

In at least one embodiment where the computing device (e.g., wearable device 100) generates the above-described plurality of sleep stages associated with the user's sleep session, the computing device can use the classifier(s) described above to generate (e.g., classify, categorize, define, characterize) one or more sleep stages using certain physiological data of the user and one or more other sleep stages using other physiological data of the user. For example, in one embodiment where the computing device generates the plurality of sleep stages, the computing device can use such classifier(s) to generate (e.g., classify, categorize, define, characterize) the light sleep stage(s) and/or determine the estimated bedtime of the user using, for instance, accelerometer data (e.g., motion data) of the user. In another embodiment where the computing device generates the plurality of sleep stages, the computing device can use such classifier(s) to generate (e.g., classify, categorize, define, characterize) the light sleep stage(s), the deep sleep stage(s), and/or the REM sleep stage(s) using, for instance, accelerometer data (e.g., motion data), heart rate data (e.g., pulse-related data), and/or respiratory data (e.g., breathing data) of the user.

According to at least one embodiment of the present disclosure, based at least in part on (e.g., in response to) obtaining the plurality of sleep stages associated with the user's sleep session, the computing device (e.g., wearable device 100) can calculate the TBSS metric based at least in part on (e.g., using) the estimated bedtime of the user and a start time of the one or more defined sleep stages. In this or another embodiment, to calculate the TBSS metric, the computing device can calculate a difference in time between the estimated bedtime of the user and the start time of the one or more defined sleep stages. For example, in this or anther embodiment, to calculate the TBSS metric, the computing device can subtract the estimated bedtime of the user from the start time of the one or more defined sleep stages to determine the difference in time between the estimated bedtime of the user and the start time of the one or more defined sleep stages.

In embodiments where the one or more defined sleep stages constitute and/or include a defined quantity of one or more of the sleep stages in the plurality of sleep stages as described above, the computing device (e.g., wearable device 100) can calculate the TBSS metric by subtracting the estimated bedtime of the user from a start time of the defined quantity of such sleep stage(s) in the plurality of sleep stages. For instance, in an embodiment where the one or more defined sleep stages constitute and/or include, for example, a bout of 20 or more consecutive, uninterrupted light sleep stages (e.g., a bout of 20 or more minutes of consecutive, uninterrupted light sleep), the computing device can calculate the TBSS metric by subtracting the estimated bedtime of the user from a start time of the bout of 20 or more consecutive, uninterrupted light sleep stages. For example, in this embodiment, the computing device can calculate the TBSS metric by subtracting the estimated bedtime of the user from a start time of the 1^{st} light sleep stage in the bout of 20 or more consecutive. uninterrupted light sleep stages.

In another embodiment where the one or more defined sleep stages constitute and/or include, for example, 1 deep sleep stage, the computing device can calculate the TBSS metric by subtracting the estimated bedtime of the user from a start time of the 1^{st} deep sleep stage in the user's sleep session. In another embodiment where the one or more defined sleep stages constitute and/or include, for example, 1 REM sleep stage, the computing device can calculate the TBSS metric by subtracting the estimated bedtime of the user from a start time of the 1^{st} REM sleep stage in the user's sleep session.

As described above, in example embodiments of the present disclosure, based at least in part (e.g., in response to) calculating the TBSS metric, the computing device (e.g., wearable device 100) can perform one or more operations based at least in part on (e.g., using) the TBSS metric. For instance, in one embodiment, based at least in part (e.g., in response to) calculating the TBSS metric, the computing device can, for example, generate an intelligent notification (e.g., a visual and/or audio notification) that can include and/or be indicative of the TBSS metric. In this or another embodiment, the computing device can further provide such an intelligent notification to the user and/or another computing device (e.g., a different computing device that is external to the computing device described above). For instance, in one embodiment, the computing device can provide the intelligent notification and/or the TBSS metric to the user using one or more data output devices such as, for example, a display device (e.g., a monitor, screen, display) and/or a speaker that can be included in, coupled to, and/or otherwise associated with the computing device.

In another embodiment described herein, the computing device (e.g., wearable device 100) can provide the above-described intelligent notification and/or the TBSS metric to another computing device (e.g., an external and/or remote computing device) such as, for instance, another client computing device, another computer, another laptop, another tablet, another smartphone, another physiological monitoring device, another wearable computing device, another wearable physiological monitoring device (e.g., another wrist-worn device, another chest strap device). In some embodiments, the computing device can provide the intelligent notification and/or the TBSS metric to another computing device and/or computing entity (e.g., module, model, algorithm, agent) that can function as and/or be associated with a medical and/or sleep counseling professional (e.g., a medical doctor, psychiatrist, sleep counselor).

In at least one embodiment of the present disclosure, based at least in part (e.g., in response to) calculating the TBSS metric, the computing device (e.g., wearable device 100) can, for example, generate one or more sleep quality recommendations based at least in part on (e.g., using) the TBSS metric. For example, in this or another embodiment, the computing device can use the TBSS metric to determine (e.g., calculate) a suggested wind down time when the user should begin to wind down, relax, and/or otherwise prepare to sleep to ensure the user can fall asleep by a certain time. In this or another embodiment, the computing device can further provide an intelligent notification (e.g., a visual and/or audio notification) that can include and/or be indicative of the TBSS metric and/or the one or more sleep quality recommendations to the user and/or another computing device (e.g., a different computing device that is external to the computing device described above). For instance, in this or another embodiment, the computing device can provide, to the user and/or another computing device, an intelligent notification that can include and/or be indicative of the TBSS metric and/or the above-described suggested wind down time in the same manner as described above.

In at least one embodiment described herein, based at least in part (e.g., in response to) calculating the TBSS metric, the computing device (e.g., wearable device 100) can, for example, implement and/or facilitate implementation of one or more sleep promoting features of the computing device and/or another computing device (e.g., a different computing device that is external to the computing device described above) based at least in part on the TBSS metric. For instance, in this or another embodiment, the computing device can implement and/or facilitate implementation of one or more sleep promoting features of the computing device and/or another computing device at the suggested wind down time described above when the user should begin to wind down, relax, and/or otherwise prepare to sleep to ensure the user can fall asleep by a certain time.

In one embodiment of the present disclosure, the computing device (e.g., wearable device 100) can implement (e.g., initiate, run, operate) one or more sleep promoting features that can be included with the computing device such as, for instance, a sleep promoting audio feature (e.g., by playing sleep promoting music and/or sounds), a sleep promoting lighting feature (e.g., by initiating a “sleep mode” and/or “night mode” of the computing device to dim one or more light sources of the computing device such as a screen, display, or monitor), and/or another sleep promoting feature of the computing device. For example, in this or another embodiment, the computing device can cause an audio system of the computing device to play sleep promoting music and/or sounds and/or cause a lighting system of the computing device to initiate a “sleep mode” and/or “night mode” to dim one or more light sources of the computing device such as a screen, display, or monitor.

In another embodiment of the present disclosure, the computing device (e.g., wearable device 100) can facilitate implementation of one or more sleep promoting features of another computing device such as, for instance: a sleep promoting audio feature of a smart audio system (e.g., a home audio system included in, coupled to, and/or operated by another computing device); a sleep promoting lighting feature of a smart lighting system (e.g., a home lighting system included in, coupled to, and/or operated by another computing device); a sleep promoting ambient temperature feature of a smart heating, ventilation, and air conditioning (HVAC) system (e.g., a home HVAC system coupled to and/or operated by another computing device); and/or another sleep promoting feature of another computing device. For instance, in this or another embodiment, the computing device can send instructions to one or more of the above-described smart systems that, when executed by such system(s) (e.g., via one or more processors), can cause the system(s) to perform operations to implement one or more sleep promoting features of such system(s).

In one embodiment of the present disclosure, the computing device (e.g., wearable device 100) can send instructions to the above-described smart audio system that, when executed by such a system (e.g., via one or more processors), can cause it to play sleep promoting music and/or sounds. In another embodiment, the computing device can send instructions to the above-described smart lighting system that, when executed by such a system (e.g., via one or more processors), can cause it to initiate a “sleep mode” and/or “night mode” to dim one or more light sources (e.g., light bulbs) of the smart lighting system. In another embodiment, the computing device can send instructions to the above-described smart HVAC system that, when executed by such a system (e.g., via one or more processors), can cause it to output air at a certain sleep promoting temperature (e.g., a certain temperature that can be defined by the user).

In at least one embodiment described herein, the computing device can (e.g., wearable device 100) record, in a database (e.g., in a log that can be stored on a memory device), the TBSS metric and/or one or more additional TBSS metrics corresponding to the user. In this embodiment, the one or more additional TBSS metrics can be calculated (e.g., by the computing device as described above) based at least in part on (e.g., using) at least one additional plurality of sleep stages associated with one or more additional sleep sessions of the user. In some embodiments, the computing device can obtain and/or record, in such a database, one or more other TBSS metrics corresponding respectively to one or more other users. In these or other embodiments, the computing device can compare the TBSS metric and/or the additional TBSS metric(s) of the user to the other TBSS metric(s) corresponding respectively to the other user(s). In these or other embodiments, the computing device can further classify the user in a defined sleep pattern category (e.g., an insomnia sleep pattern category) based at least in part on comparison of the TBSS metric and/or the additional TBSS metric(s) of the user to the other TBSS metric(s) corresponding respectively to the other user(s). In some embodiments, to perform the comparison and/or classification operations described above, the computing system can use one or more of the above-described classifiers and/or another classifier that can compare one or more TBSS metrics of the user with one or more TBSS metrics of one or more other users and classify the user in a defined sleep pattern category based on such comparison.

In at least one embodiment of the present disclosure, the computing device (e.g., wearable device 100) can identify a defined sleep pattern of the user based at least in part on the TBSS metric and/or the additional TBSS metrics corresponding to the user. For instance, in this or another embodiment, by comparing one or more TBSS metrics of the user with one or more TBSS metrics of one or more other users and classifying the user in a defined sleep pattern category based on such comparison as described above (e.g., via one or more classifiers), the computing device can thereby determine that the user's sleep pattern corresponds to a certain sleep pattern such as, for example, an insomnia sleep pattern. In some embodiments, based at least in part on (e.g., in response to) identifying such a defined sleep pattern of the user, the computing device can further determine a defined sleep condition diagnosis and/or a defined sleep condition prognosis that can be associated with sleep quality of the user. For instance, in these or other embodiments, based at least in part on (e.g., in response to) determining the user's sleep pattern corresponds to an insomnia sleep pattern as described above, the computing device can further diagnose the user as an insomniac.

Example aspects of the present disclosure provide several technical effects, benefits, and/or improvements in computing technology. For instance, according to example embodiments the present disclosure, a computing device such as, for instance, wearable device 100 can calculate and use one or more TBSS metrics of a user as described herein to accurately and consistently determine when the user is in a relaxing state of stillness (e.g., watching television in bed, reading) and when the user is in a true sleep state (e.g., when the user enters the above-defined sound sleep state).

In some embodiments, by calculating and using the TBSS metric(s) of a user as described herein to accurately and consistently determine when the user is in a relaxing state of stillness (e.g., watching television in bed, reading) and when the user is in a true sleep state (e.g., when the user enters the above-defined sound sleep state), the above-described computing device (e.g., wearable device 100) can thereby reduce the processing workload of one or more processors that execute operations to make such a determination. For example, in these or other embodiments, the above-described computing device can thereby reduce the processing workload of one or more processors that can be included in and/or coupled to the computing device and/or another computing device that is external to the computing device such as, for instance, another computing device and/or computing entity (e.g., module, model, algorithm, agent) that can function as and/or be associated with a medical and/or sleep counseling professional (e.g., a processor of another computing device that can be used to conduct sleep studies, diagnosis patients with various sleep conditions, and/or treat patients having such sleep conditions). In these or other embodiments, by reducing the processing workload of such one or more processors, the computing device can thereby improve the processing efficiency and/or processing performance of the processor(s), as well as reduce computational costs of the processor(s).

### Example Devices and Systems

FIGS. 1, 2, and 3 each illustrate a perspective view of an example, non-limiting wearable device 100 according to one or more example embodiments of the present disclosure. In example embodiments described herein, wearable device 100 can constitute and/or include a wearable computing device. For instance, in these or other example embodiments, wearable device 100 can constitute and/or include a wearable computing device such as, for example, a wearable physiological monitoring device that can be worn by a user (also referred to herein as a “wearer”) and/or capture one or more types of physiological data of the user (e.g., heart rate data, pulse-based data, motion data, temperature data).

Wearable device 100 according to example embodiments of the present disclosure can include a display 102, an attachment component 104, a securement component 106, and a button 108 that can be located on a side of wearable device 100. In at least one embodiment, two sides of display 102 can be coupled (e.g., mechanically, operatively) to attachment component 104. In some embodiments, securement component 106 can be located on, coupled to (e.g., mechanically, operatively), and/or integrated with attachment component 104. In these or other embodiments, securement component 106 can be positioned opposite display 102 on an opposing end of attachment component 104. In some embodiments, button 108 can be located on a side of wearable device 100, underneath display 102.

Display 102 according to example embodiments described herein can constitute and/or include any type of electronic display or screen known in the art. For example, in some embodiments, display 102 can constitute and/or include a liquid crystal display (LCD) or organic light emitting diode (OLED) display such as, for instance, a transmissive LCD display or a transmissive OLED display. Display 102 according to example embodiments can be configured to provide brightness, contrast, and/or color saturation features according to display settings that can be maintained by control circuitry and/or other internal components and/or circuitry of wearable device 100. In some embodiments, display 102 can constitute and/or include a touchscreen such as, for instance, a capacitive touchscreen. For example, in these embodiments, display 102 can constitute and/or include a surface capacitive touchscreen or a projective capacitive touch screen that can be configured to respond to contact with electrical charge-holding members or tools, such as a human finger.

In some embodiments, display 102 can be configured to provide (e.g., render) a variety of information such as, for example, the time, the date, body signals (e.g., physiological data of a user wearing wearable device 100), readings based upon user input, and/or other information. In one embodiment, such body signals can include, but are not limited to, heart rate data (e.g., heart beats per minute), pulse-rate data, motion data (e.g., movement data), blood pressure data, temperature data, oxygen levels data, and/or any other body signal that one of ordinary skill in the art would understand that can be measured by a wearable device such as, for instance, wearable device 100. In some embodiments, the readings based upon user input can include, but are not limited to, the number of steps a user has taken, the distance traveled by the user, the sleep schedule of the user, travel routes of the user, elevation climbed by the user, and/or any other metric that one of ordinary skill in the art would understand that can be input by a user into a wearable device such as, for instance, wearable device 100.

In at least one embodiment of the present disclosure, the above-described body signals and/or readings based upon user input can be used to calculate further analytics to provide a user with data such as, for instance, a fitness score, a sleep quality score, the TBSS metric described herein, a number of calories burned by the user, and/or other data. In some embodiments, wearable device 100 can take in (e.g., capture, collect, receive, measure) outside data irrespective of the user such as, for example: an ambient temperature of an environment surrounding and/or external to wearable device 100; an amount of sun exposure wearable device 100 is subjected to; an atmospheric pressure of the environment surrounding and/or external to wearable device 100; an air quality of the environment surrounding and/or external to wearable device 100; the location of wearable device 100 based on, for instance, a global positioning system (GPS); and/or other outside factors that one of ordinary skill in the art would understand a wearable device such as, for instance, wearable device 100 can take in (e.g., capture, collect, receive, measure).

Attachment component 104 according to example embodiments described herein can be used to attach (e.g., affix, fasten) wearable device 100 to a user of wearable device 100. In some embodiments, attachment component 104 can take the form of, for example, a strap, an elastic band, a rope, and/or any other form of attachment one of ordinary skill in the art would understand can be used to attach a wearable device such as, for instance, wearable device 100 to a user.

Securement component 106 according to example embodiments of the present disclosure can facilitate attachment of attachment component 104 upon a user of wearable device 100. In some embodiments, securement component 106 can include, but is not limited to, a pin and hole locking mechanism (e.g., a buckle), a magnet system, a lock, a clip, and/or any other type of securement that one of ordinary skill would understand can be used to facilitate attachment of a wearable device such as, for instance, wearable device 100 to a user. In one embodiment, wearable device 100 does not include securement component 106. For example, in this or another embodiment, wearable device 100 can be secured to a user with a strap that can be tied around the user's wrist and/or another suitable appendage.

Button 108 according to example embodiments described herein can allow for a user to interact with wearable device 100 and/or allow for the user to provide a form of input into wearable device 100. In the example embodiment depicted in FIGS. 1, 2, and 3, one button 108 is shown on wearable device 100. However, it should be appreciated that wearable device 100 is not so limiting. For example, in some embodiments, wearable device 100 can include any number of buttons that allow a user to further interact with wearable device 100 and/or to provide alternative inputs. In at least one embodiment, wearable device 100 does not include button 108. For instance, as described above, in example embodiments, wearable device 100 can include a screen such as, for example, a touch screen that can receive inputs through (e.g., by way of) the touch of the user. In additional or alternative embodiments, wearable device 100 can include a microphone that can receive inputs through (e.g., by way of) voice commands of a user.

In some embodiments, wearable device 100 can constitute a portable computing device that can be designed so that it can be inserted into a wearable case (e.g., as illustrated in the example embodiments depicted in FIGS. 1, 2, and 3). In some embodiments, wearable device 100 can constitute a portable computing device that can be designed so that it can be inserted into one or more of multiple different wearable cases (e.g., a wristband case, a belt-clip case, a pendant case, a case configured to be attached to a piece of exercise equipment such as a bicycle). Wearable device 100 according to embodiments described herein can be formed into one or more shapes and/or sizes to allow for coupling to (e.g., secured to, worn, borne by) the body or clothing of a user. In some embodiments, wearable device 100 can constitute a portable computing device that can be designed to be worn in limited manners such as, for instance, a computing device that is integrated into a wristband in a non-removable manner and/or can be intended to be worn specifically on a person's wrist (or perhaps ankle).

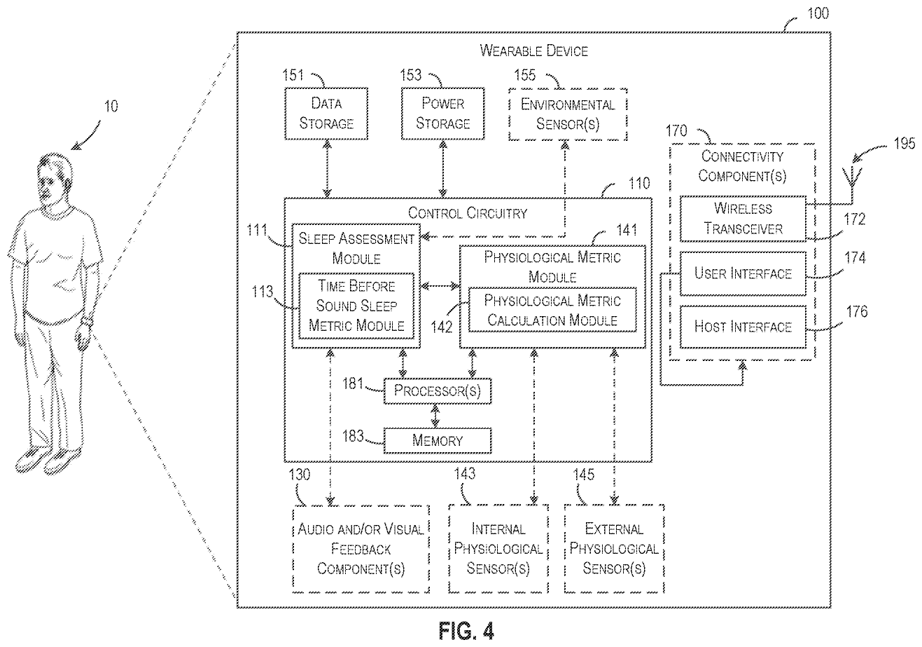

Irrespective of configuration, wearable device 100 according to example embodiments of the present disclosure can include one or more physiological and/or environmental sensors (e.g., internal physiological sensor(s) 143, external physiological sensor(s) 145, and/or environmental sensor(s) 155 described below with reference to FIG. 4) that can be configured to collect physiological and/or environmental data in accordance with various embodiments disclosed herein. In some embodiments, wearable device 100 can be configured to analyze and/or interpret collected physiological and/or environmental data to perform a sleep quality assessment (e.g., by calculating the above-described TBSS metric according to example embodiments described herein) of a user (e.g., wearer) of wearable device 100, or can be configured to communicate with another computing device or server that can perform the sleep quality assessment (e.g., by calculating the above-described TBSS metric according to example embodiments described herein).

Wearable device 100 in accordance with one or more example embodiments of the present disclosure can include one or more physiological and/or environmental components and/or modules that can be designed to determine one or more physiological and/or environmental metrics associated with a user (e.g., a wearer) of wearable device 100. In at least one embodiment, such physiological and/or environmental component(s) and/or module(s) can constitute and/or include one or more physiological and/or environmental sensors. For instance, although not depicted in the example embodiments illustrated in FIGS. 1, 2, and 3, in some embodiments, wearable device 100 can include one or more physiological and/or environmental sensors such as, for example, an accelerometer, a heart rate sensor (e.g., photoplethysmography (PPG) sensor), a body temperature sensor, an environment temperature sensor, and/or another physiological and/or environmental sensor. In these or other embodiments, such physiological and/or environmental sensor(s) can be disposed on, coupled to, and/or otherwise be associated with an underside and/or a backside (e.g., back 134) of wearable device 100.

In some embodiments, the above-described physiological and/or environmental sensor(s) can be disposed on, coupled to, and/or otherwise be associated with wearable device 100 such that the sensor(s) can be in contact with or substantially in contact with human skin when wearable device 100 is worn by a user. For example, in embodiments where wearable device 100 can be worn on a user's wrist, the physiological and/or environmental sensor(s) can be disposed on, coupled to, and/or otherwise be associated with back 134 that can be substantially opposite display 102 and touching an arm of the user. In one embodiment, the above-described physiological and/or environmental sensor(s) can be disposed on, coupled to, and/or otherwise be associated with an interior or skin-side of wearable device 100 (e.g., a side of wearable device 100 that contacts, touches, and/or faces the skin of the user such as, for instance, back 134 and/or bottom 142). In another embodiment, the physiological and/or environmental sensors can be disposed on one or more sides of wearable device 100, including the skin-side (e.g., back 134, bottom 142) and one or more sides (e.g., first side 136, second side 138, top 140, display 102) of wearable device 100 that face and/or are exposed to the ambient environment (e.g., the external environment surrounding wearable device 100).

FIG. 4 illustrates a block diagram of the above-described example, non-limiting wearable device 100 according to one or more example embodiments of the present disclosure. That is, for instance, FIG. 4 illustrates a block diagram of one or more internal and/or external components of the above-described example, non-limiting wearable device 100 according to one or more example embodiments of the present disclosure.

As described above with reference to the example embodiments depicted in FIGS. 1, 2, and 3, wearable device 100 can constitute and/or include a wearable computing device such as, for instance, a wearable physiological monitoring device. For example, in the example embodiment depicted in FIG. 4, wearable device 100 can constitute and/or include a wearable physiological monitoring device that can be worn by a user 10 (also referred to herein as a “wearer” or “wearer 10”) and/or can be configured to gather data regarding activities performed by user 10 and/or regarding user's 10 physiological state. In this or another embodiment, such data can include data representative of the ambient environment around user 10 or user's 10 interaction with the environment. For example, in some embodiments, the data can constitute and/or include motion data regarding user's 10 movements, ambient light, ambient noise, air quality, and/or physiological data obtained by measuring various physiological characteristics of user 10 (e.g., heart rate, pulse-based data, respiratory data, body temperature, blood oxygen levels, perspiration levels).

Although certain embodiments are disclosed herein in the context of wearable physiological monitoring devices, it should be appreciated that the present disclosure is not so limiting. For example, it should be understood that the physiological monitoring and sleep quality assessment principles and features disclosed herein can be applicable with respect to and/or implemented using any suitable or desirable type of computing device or combination of computing devices, whether wearable or not. For instance, the calculation and/or application of the TBSS metric described herein in accordance with one or more embodiments can by performed and/or implemented using any suitable or desirable type of computing device or combination of computing devices such as, for example, a client computing device, a laptop, a tablet, a server, a wearable computing device, and/or another computing device, whether wearable or not.

As illustrated in FIG. 4, wearable device 100 according to example embodiments of the present disclosure can include one or more audio and/or visual feedback components 130 such as, for instance, electronic touchscreen display units, light-emitting diode (LED) display units, audio speakers, light-emitting diode (LED) lights, buzzers, and/or another type of audio and/or visual feedback module. In certain embodiments, one or more audio and/or visual feedback modules 130 can be located on and/or otherwise associated with a front side of wearable device 100 and/or display 102. For example, in wearable embodiments of wearable device 100, an electronic display such as, for instance, display 102 can be configured to be externally presented to user 10 viewing wearable device 100.

Wearable device 100 according to example embodiments of the present disclosure can include control circuitry 110. Although certain modules and/or components are illustrated as part of control circuitry 110 in the diagram of FIG. 4, it should be understood that control circuitry 110 associated with wearable device 100 and/or other components or devices in accordance with example embodiments of the present disclosure can include additional components and/or circuitry such as, for instance, one or more additional components of the illustrated components depicted in FIG. 4. Furthermore, in certain embodiments, one or more of the illustrated components of control circuitry 110 can be omitted and/or different than that shown in FIG. 4 and described in association therewith.

The term “control circuitry” is used herein according to its broad and/ordinary meaning and can include any combination of software and/or hardware elements, devices, and/or features that can be implemented in connection with operation of wearable device 100. Furthermore, the term “control circuitry.” can be used substantially interchangeably in certain contexts herein with one or more of the terms “controller,” “integrated circuit,” “IC,” “application-specific integrated circuit,” “ASIC,” “controller chip,” or the like.

Control circuitry 110 according to example embodiments of the present disclosure can constitute and/or include one or more processors, data storage devices, and/or electrical connections. In one embodiment, control circuitry 110 can be implemented on a system on a chip (SoC), however, those skilled in the art will recognize that other hardware and/or firmware implementations are possible.

In one or more embodiments of the present disclosure, control circuitry 110 can constitute and/or include one or more processors 181 that can be configured to execute computer-readable instructions that, when executed, cause wearable device 100 to perform one or more operations. In at least one embodiment, control circuitry 110 can constitute and/or include processor(s) 181 that can be configured to execute operational code (e.g., instructions, processing threads, software) for wearable device 100 such as, for instance, firmware or the like. Processor(s) 181 according to example embodiments described herein can each be a processing device. For instance, in the example embodiment depicted in FIG. 4, processor(s) 181 can each be a central processing unit (CPU), microprocessor, microcontroller, integrated circuit (e.g., an application-specific integrated circuit (ASIC)), and/or another type of processing device. In this or another example embodiment, processor(s) 181 can be coupled to (e.g., electrically, communicatively, physically, operatively) to one or more components of control circuitry 110 and/or wearable device 100 such that processor(s) 181 can facilitate one or more operations in accordance with one or more example embodiments described herein.

In at least one embodiment of the present disclosure, the above-described computer-readable instructions and/or operational code that can be executed by processor(s) 181 can be stored in one or more data storage devices of wearable device 100. In the example embodiment depicted in FIG. 4, such computer-readable instructions and/or operational code can be stored in memory 183 of wearable device 100. In this or another example embodiment, memory 183 can be coupled to (e.g., electrically, communicatively, physically, operatively) to one or more components of control circuitry 110 and/or wearable device 100 such that memory 183 can facilitate one or more operations in accordance with one or more example embodiments described herein.

Memory 183 according to example embodiments described herein can store computer-readable and/or computer executable entities (e.g., data, information, applications, models, algorithms) that can be created, modified, accessed, read, retrieved, and/or executed by each of processor(s) 181. In some embodiments, memory 183 can constitute, include, be coupled to (e.g., operatively), and/or otherwise be associated with a computing system and/or media such as, for example, one or more computer-readable media, volatile memory, non-volatile memory, random-access memory (RAM), read only memory (ROM), hard drives, flash drives, and/or other memory devices. In these or other embodiments, such one or more computer-readable media can include, constitute, be coupled to (e.g., operatively), and/or otherwise be associated with one or more non-transitory computer-readable media. Although not depicted in the example embodiment illustrated in FIG. 4, in some embodiments, memory 183 can include (e.g., store) sleep assessment module 111, TBSS metric module 113, physiological metric module 141, physiological metric calculation module 142, and/or other modules and/or data that can be used to facilitate one or more operations described herein.

Control circuitry 110 according to example embodiments of the present disclosure can constitute and/or include a sleep assessment module 111. Sleep assessment module 111 according to example embodiments of the present disclosure can constitute and/or include one or more hardware and/or software components and/or features that can be configured to make an assessment of sleep quality of user 10, optionally using inputs from one or more environmental sensors 155 (e.g., ambient light sensor) and/or information from physiological metric module 141. In certain embodiments, sleep assessment module 111 can include a time before sound sleep metric module 113 (also referred to herein as “TBSS metric module 113”) that can be configured to calculate the above-described TBSS metric. For example, in these or other embodiments, wearable device 100 can implement TBSS metric module 113 to calculate the TBSS metric for user 10 as described herein in accordance with one or more embodiments of the present disclosure.

In one embodiment, wearable device 100 can implement TBSS metric module 113 to calculate the TBSS metric for user 10 by obtaining the above-described plurality of sleep stages and using such sleep stages to calculate the TBSS metric in accordance with one or more embodiments described herein. In another embodiment, wearable device 100 can implement TBSS metric module 113 to calculate the TBSS metric for user 10 by generating the above-described plurality of sleep stages and using such sleep stages to calculate the TBSS metric in accordance with one or more embodiments described herein. For instance, in this embodiment, wearable device 100 can use the above-described classifier(s) to generate the plurality of sleep stages and use such sleep stages to calculate the TBSS metric in accordance with one or more embodiments described herein. In this embodiment, wearable device 100 can implement the classifier(s) to generate the plurality of sleep stages using physiological data of user 10 that can be accumulated by sleep assessment module 111 such as, for instance, the values of one or more physiological metrics (e.g., user's 10 heart rate, motion, temperature, respiration) that can be determined by physiological metric calculation module 142 of physiological metric module 141.

In certain embodiments, physiological metric module 141 and/or physiological metric calculation module 142 can be communicatively coupled with one or more internal physiological sensors 143 that can be embedded and/or integrated in wearable device 100. In certain embodiments, physiological metric module 141 and/or physiological metric calculation module 142 can be optionally in communication with one or more external physiological sensors 145 not embedded and/or integrated in wearable device 100 (e.g., an electrode or sensor integrated in another electronic device). In some embodiments, examples of internal physiological sensors 143 and/or external physiological sensors 145 can constitute and/or include, but are not limited to, one or more sensors that can measure (e.g., capture, collect, receive) physiological data of user 10 such as, for instance, body temperature, heart rate, blood oxygen level, movement, respiration, and/or other physiological data of user 10.

In the example embodiment depicted in FIG. 4, wearable device 100 can include one or more data storage components 151 (denoted as “data storage 151” in FIG. 4). Data storage component(s) 151 according to example embodiments can constitute and/or include any suitable or desirable type of data storage such as, for instance, solid-state memory, which can be volatile or non-volatile. In some embodiments, such solid-state memory of wearable device 100 can constitute and/or include any of a wide variety of technologies such as, for instance, flash integrated circuits, phase change (PC) memory, phase change (PC) random-access memory (RAM), programmable metallization cell RAM (PMC-RAM or PMCm), ovonic unified memory (OUM), resistance RAM (RRAM), NAND memory, NOR memory, EEPROM, ferroelectric memory (FeRAM), MRAM, or other discrete NVM (non-volatile solid-state memory) chips. In some embodiments, data storage component(s) 151 can be used to store system data, such as operating system data and/or system configurations or parameters. In some embodiments, wearable device 100 can include data storage utilized as a buffer and/or cash memory for operational use by control circuitry 110.

Data storage component(s) 151 according to example embodiments can include various sub-modules such as, for instance, one or more of: a sleep detection module (e.g., sleep assessment module 111, TBSS metric module 113) that can detect an attempt or onset of sleep by the user 10; an information collection module (e.g., physiological metric module 141, physiological metric calculation module 142) that can manage the collection of physiological and/or environmental data relevant to a sleep quality assessment; a sleep quality metric calculation module (e.g., sleep assessment module 111, TBSS metric module 113) that can determine values of one or more sleep quality metrics as described in the present disclosure; a unified score determination module that can determine a representation of a unified sleep quality score as described in the present disclosure; a presentation module that can manage presentation of sleep quality assessment information to user 10; a heart rate determination module that can determine values and/or patterns of one or more types of heart rates of user 10; a feedback management module for collecting and interpreting sleep quality feedback from user 10; and/or another sub-module.

Wearable device 100 according to example embodiments can further include a power storage module 153 (denoted as “power storage 153”), which can constitute and/or include a rechargeable battery, one or more capacitors, or other charge-holding device(s). In some embodiments, the power stored by power storage module 153 can be utilized by control circuitry 110 for operation of wearable device 100, such as for powering display 102. In some embodiments, power storage module 153 can receive power over a host interface of wearable device 100 (e.g., via one or more host interface circuitry and/or components 176 (denoted as “host interface 176” in FIG. 4)) and/or through other means.

Wearable device 100 according to example embodiments can further include one or more environmental sensors 155. In at least one embodiment, examples of such environmental sensors 155 can include, but are not limited to, sensors that can determine and/or measure, for instance, ambient light, external (non-body) temperature, altitude, device location (e.g., global-positioning system (GPS)), and/or another environmental data.

Wearable device 100 according to example embodiments can further include one or more connectivity components 170, which can include, for example, a wireless transceiver 172. Wireless transceiver 172 according to example embodiments can be communicatively coupled to one or more antenna devices 195, which can be configured to wirelessly transmit and/or receive data and/or power signals to and/or from wearable device 100 using, but not limited to, peer-to-peer, WLAN, and/or cellular communications. For example, wireless transceiver 172 can be utilized to communicate data and/or power between wearable device 100 and an external computing device (not illustrated in FIG. 4) such as, for instance, an external client computing device (e.g., a smartphone, tablet, computer) and/or an external host system (e.g., a server), which can be configured to interface with wearable device 100. In certain embodiments, wearable device 100 can include one or more host interface circuitry and/or components 176 (denoted as “host interface 176” in FIG. 4) such as, for instance, wired interface components that can communicatively couple wearable device 100 with the above-described external computing device (e.g., a smartphone, table, computer, server) to receive data and/or power therefrom and/or transmit data thereto.

Connectivity component(s) 170 according to example embodiments can further include one or more user interface components 174 (denoted as “user interface 174” in FIG. 4) that can be used by wearable device 100 to receive input data from user 10 and/or provide output data to user 10. In some embodiments, user interface component(s) 174 can be coupled to (e.g., operatively, communicatively) and/or otherwise be associated with audio and/or visual feedback component(s) 130. For instance, in these embodiments, display 102 of wearable device 100 can constitute and/or include a touchscreen display that can be configured to provide (e.g., render) output data to user 10 and/or to use audio and/or visual feedback component(s) 130 to receive user input through user contact with the touchscreen display. In some embodiments, user interface component(s) 174 can further constitute and/or include one or more buttons or other input components or features.

Connectivity component(s) 170 according to example embodiments can further include host interface circuitry and/or component(s) 176, which can be, for example, an interface that can be used by wearable device 100 to communicate with the above-described external computing device (e.g., a smartphone, table, computer, server) over a wired or wireless connection. Host interface circuitry and/or component(s) 176 according to example embodiments can utilize and/or otherwise be associated with any suitable or desirable communication protocol and/or physical connector such as, for instance, universal serial bus (USB), micro-USB, Wi-Fi, Bluetooth, FireWire, PCIe, or the like. For wireless connections, host interface circuitry and/or component(s) 176 according to example embodiments can be incorporated with wireless transceiver 172.

Although certain functional modules and components are illustrated and described herein, it should be understood that authentication management functionality in accordance with the present disclosure can be implemented using a number of different approaches. For example, in some embodiments, control circuitry 110 can constitute and/or include one or more processors (e.g., processor(s) 181) that can be controlled by computer-executable instructions that can be stored in a memory (e.g., memory 183, data storage component(s) 151) so as to provide functionality such as is described herein. In other embodiments, such functionality can be provided in the form of one or more specially designed electrical circuits. In some embodiments, such functionality can be provided by one or more processors (e.g., processor(s) 181) that can be controlled by computer-executable instructions that can be stored in a memory (e.g., memory 183, data storage component(s) 151) that can be coupled to (e.g., communicatively, operatively, electrically) one or more specially designed electrical circuits. Various examples of hardware that can be used to implement the concepts outlined herein can include, but are not limited to, application specific integrated circuits (ASICs), field-programmable gate arrays (FPGAs), and general-purpose microprocessors that can be coupled with memory that stores executable instructions for controlling the general-purpose microprocessors.

FIG. 5 illustrates a diagram of an example, non-limiting sleep quality management system 500 according to one or more example embodiments of the present disclosure. Sleep quality management system 500 depicted in FIG. 5 illustrates an example, non-limiting networked relationship between wearable device 100, an external computing device 504, and/or one or more smart systems 512 in accordance with one or more embodiments.

As described above, wearable device 100 according to example embodiments of the present disclosure can assess sleep quality of user 10 and/or facilitate alteration (e.g., improvement) of user's 10 sleep quality based on such assessment. More specifically, wearable device 100 according to example embodiments described herein can calculate the TBSS metric associated with user 10 and further use the TBSS metric to perform one or more operations that can facilitate alteration (e.g., improvement) of user's 10 sleep quality. As such, in certain embodiments described in the present disclosure, wearable device 100 can be capable of and/or configured to collect physiological sensor readings of user 10 and/or calculate the TBSS metric using such readings.

However, in additional and/or alternative embodiments, wearable device 100, or another electronic and/or computing device that can be used to detect physiological information of user 10, can be in communication with external computing device 504. In these or other embodiments, external computing device 504 can be configured to use such physiological information of user 10 to calculate user's 10 TBSS metric (e.g., by obtaining or generating the above-described plurality of sleep stages and using such sleep stages to calculate the TBSS metric in accordance with one or more embodiments described herein). In these or other embodiments, external computing device 504 can further use user's 10 TBSS metric to perform one or more operations that can facilitate alteration (e.g., improvement) of user's 10 sleep quality.

Wearable device 100 according to example embodiments can be configured to collect one or more types of physiological and/or environmental data using embedded sensors and/or external devices, as described throughout the present disclosure, and communicate or relay such information over one or more networks 506 to other devices. This includes, in some embodiments, relaying information to devices capable of serving as Internet-accessible data sources, thus permitting the collected data to be viewed, for example, using a web browser or network-based application at, for instance, external computing device 504. For example, while user 10 is attempting to sleep and/or is asleep and is wearing wearable device 100, wearable device 100 can calculate and optionally store user's 10 heart rate, motion data, temperature, and/or respiration using one or more physiological sensors. Wearable device 100 according to example embodiments can then transmit data representative of user's 10 heart rate, motion data, temperature, and/or respiration over network(s) 506 to an account on a web service, computer, mobile phone, and/or health station where the data can be stored, processed, and visualized by user 10 and/or another entity (e.g., a health care professional).

While wearable device 100 is shown in example embodiments of the present disclosure to have a display, it should be understood that, in some embodiments, wearable device 100 does not have any type of display unit. In some embodiments, wearable device 100 can have audio and/or visual feedback components such as, for instance, light-emitting diodes (LEDs), buzzers, speakers, and/or a display with limited functionality. Wearable device 100 according to example embodiments can be configured to be attached to user's 10 body or clothing. For example, in these or other embodiments, wearable device 100 can be configured as a wrist bracelet, watch, ring, electrode, finger-clip, toe-clip, chest-strap, ankle strap, and/or a device placed in a pocket. In additional or alternative embodiments, wearable device 100 can be embedded in something in contact with user 10 such as, for instance, clothing, a mat that can be positioned under user 10, a blanket, a pillow, and/or another accessory involved in the activity of engaging in sleep.

In one or more embodiments of the present disclosure, the communication between wearable device 100 and external computing device 504 can be facilitated by network(s) 506. In some embodiments, network(s) 506 can constitute and/or include, for instance, one or more of an ad hoc network, a peer-to-peer communication link, an intranet, an extranet, a virtual private network (VPN), a local area network (LAN), a wireless LAN (WLAN), a wide area network (WAN), a wireless WAN (WWAN), a metropolitan area network (MAN), a portion of the Internet, a portion of the public switched telephone network (PSTN), a cellular telephone network, and/or any other type of network. In some embodiments, the communication between wearable device 100 and external computing device 504 can also be performed through a direct wired connection. In these or other embodiments, this direct-wired connection can be associated with any suitable or desirable communication protocol and/or physical connector such as, for instance, universal serial bus (USB), micro-USB, Wi-Fi, Bluetooth, FireWire, PCIe, or the like.

In example embodiments of the present disclosure, a variety of computing devices can be in communication with wearable device 100 to facilitate sleep quality assessment and/or alteration (e.g., improvement). Although external computing device 504 is depicted as a smartphone in the example embodiment illustrated in FIG. 5, it should be understood that the present disclosure is not so limiting. For instance, external computing device 504 according to example embodiments can constitute and/or include, for example, a smartphone with a display 508 as depicted in FIG. 5, a personal digital assistant (PDA), a mobile phone, a tablet, a personal computer, a laptop computer, a smart television, a video game console, a server, and/or another computing device that can be external to wearable device 100.

The networked relationship depicted in the example embodiment illustrated in FIG. 5 demonstrates how, in some embodiments, external computing device 504 can be implemented to calculate the TBSS metric associated with user 10 and/or further use the TBSS metric to perform one or more operations that can facilitate alteration (e.g., improvement) of user's 10 sleep quality. For example, in one embodiment, user 10 can wear wearable device 100 that can be equipped as a bracelet with one or more physiological sensors but without a display. In this or another embodiment, over the course of a sleep session of user 10 (e.g., over the course of the night), as user 10 attempts to fall asleep and then enters a sound sleep state as defined herein, wearable device 100 can record, for instance, user's 10 heartbeat, movement, body temperature, respiration, and/or blood oxygen level, as well as room temperature and/or ambient light levels. In this or another embodiment, wearable device 100 can periodically transmit such information to external computing device 504 over network(s) 506.

In additional and/or alternative embodiments, wearable device 100 can store the above-described collected physiological and/or environmental data and transmit this data to external computing device 504 in response to a trigger such as, for instance, detection of user 10 being awake after a period of being asleep. In some embodiments, user 10 can be awake for a threshold period of time to set this trigger (e.g., awake for at least 10 minutes), and/or awake for a threshold period of time after a threshold period of sleep has been experienced (e.g., awake for at least 5 minutes after having at least 6 hours of sleep). In some embodiments, a trigger for calculating the TBSS metric of user 10 can be detection of a command performed by external computing device 504 such as, for instance, manual or automatic execution of an instruction to synchronize collected physiological and/or environmental data and calculate the TBSS metric (e.g., by obtaining or generating the above-described plurality of sleep stages and using such sleep stages to calculate the TBSS metric in accordance with one or more embodiments described herein).

In some embodiments, external computing device 504 can present (e.g., provide, render) the TBSS metric (e.g., a time value rendered in minutes). For instance, in these or other embodiments, external computing device 504 can generate an intelligent notification 510 that can include the TBSS metric and/or one or more sleep quality recommendations (e.g., a suggested wind down time) that, if and/or when implemented by user 10, can facilitate alteration (e.g., improvement) of user's 10 sleep quality. In the example embodiment depicted in FIG. 5, external computing device 504 can render intelligent notification 510 having the TBSS metric and the sleep quality recommendation(s) on display 508 such that user 10 and/or another entity (e.g., a health care professional, a sleep therapy provider, a doctor, a caregiver) can view such information.

In some embodiments, external computing device 504 can: calculate user's 10 TBSS metric; determine one or more sleep quality recommendations based on (e.g., in response to) the TBSS metric; generate intelligent notification 510 such that it includes the TBSS metric and the sleep quality recommendation(s); and send this information back to wearable device 100 over network(s) 506 for presentation (e.g., via display 102) of such information to user 10 and/or another entity (e.g., a health care professional, a sleep therapy provider, a doctor, a caregiver). Although not illustrated in the example embodiment depicted in FIG. 5, in some embodiments, wearable device 100 can: calculate user's 10 TBSS metric; determine one or more sleep quality recommendations based on (e.g., in response to) the TBSS metric; generate intelligent notification 510 such that it includes the TBSS metric and the sleep quality recommendation(s); and render this information on display 102 of wearable device 100.

In at least one embodiment described herein, based at least in part (e.g., in response to) calculating the TBSS metric of user 10, wearable device 100 and/or external computing device 504 can, for example, implement and/or facilitate implementation of one or more sleep promoting features of wearable device 100 and/or external computing device 504 based at least in part on user's 10 TBSS metric. For instance, in this or another embodiment, wearable device 100 and/or external computing device 504 can implement and/or facilitate implementation of one or more sleep promoting features of wearable device 100 and/or external computing device 504 at a recommended wind down time that can be suggested by wearable device 100 and/or external computing device 504 (e.g., a recommended time when user 10 should begin to wind down, relax, and/or otherwise prepare to sleep to ensure user 10 can fall asleep by a certain time).

In one embodiment of the present disclosure, wearable device 100 and/or external computing device 504 can implement (e.g., initiate, run, operate) one or more sleep promoting features that can be included with wearable device 100 and/or external computing device 504 such as, for instance, a sleep promoting audio feature (e.g., by playing sleep promoting music and/or sounds), a sleep promoting lighting feature (e.g., by initiating a “sleep mode” and/or “night mode” of wearable device 100 and/or external computing device 504 to dim one or more light sources of wearable device 100 and/or external computing device 504 such as a screen, display, or monitor), and/or another sleep promoting feature of wearable device 100 and/or external computing device 504. For example, in this or another embodiment, wearable device 100 and/or external computing device 504 can cause an audio system of wearable device 100 and/or external computing device 504 to play sleep promoting music and/or sounds and/or cause a lighting system of wearable device 100 and/or external computing device 504 to initiate a “sleep mode” and/or “night mode” to dim one or more light sources of wearable device 100 and/or external computing device 504 such as a screen, display, or monitor.

In another embodiment of the present disclosure, wearable device 100 and/or external computing device 504 can facilitate implementation of one or more sleep promoting features of another computing device such as, for instance, a computing device of one or more smart systems 512. In this or another embodiment, smart system(s) 512 can constitute and/or include, but are not limited to, an audio system (e.g., a home audio system), a lighting system (e.g., a home lighting system), an HVAC system (e.g., a home HVAC system), and/or another system that can be included in, coupled to, and/or operated by a computing device other than wearable device 100 and/or external computing device 504. For instance, in some embodiments, smart system(s) 512 can constitute and/or include a smart audio system, a smart lighting system, and/or a smart HVAC system. In these or other embodiments, wearable device 100 and/or external computing device 504 can facilitate implementation of one or more sleep promoting features of smart system(s) 512 such as, for instance: a sleep promoting audio feature of a smart audio system; a sleep promoting lighting feature of a smart lighting system; a sleep promoting ambient temperature feature of a smart HVAC system; and/or another sleep promoting feature of smart system(s) 512.

In some embodiments described herein, wearable device 100 and/or external computing device 504 can send instructions to smart system(s) 512 that, when executed by such system(s) (e.g., via one or more processors), can cause the system(s) to perform operations to implement one or more sleep promoting features of such system(s). In one embodiment, wearable device 100 and/or external computing device 504 can send instructions to a smart audio system that, when executed by such a system (e.g., via one or more processors), can cause it to play sleep promoting music and/or sounds. In another embodiment, wearable device 100 and/or external computing device 504 can send instructions to a smart lighting system that, when executed by such a system (e.g., via one or more processors), can cause it to initiate a “sleep mode” and/or “night mode” to dim one or more light sources (e.g., light bulbs) of the smart lighting system. In another embodiment, wearable device 100 and/or external computing device 504 can send instructions to a smart HVAC system that, when executed by such a system (e.g., via one or more processors), can cause it to output air at a certain sleep promoting temperature (e.g., a certain temperature that can be defined by user 10).

FIG. 6 illustrates a diagram of an example, non-limiting sleep quality management system 600 according to one or more example embodiments of the present disclosure. Sleep quality management system 600 depicted in FIG. 6 illustrates an example, non-limiting networked relationship between one or more wearable devices 100a, 100b, 100c, one or more external computing devices 504a, 504b, 504c, and/or a server system 604 in accordance with one or more embodiments.

In the example embodiment depicted in FIG. 6, wearable devices 100a, 100b, 100c can each include the same characteristics, structure, components, attributes, and/or functionality as that of wearable device 100. In this embodiment, each wearable device 100a, 100b, 100c can be coupled to (e.g., worn by) a respective user 10a, 10b, 10c. In this embodiment, external computing devices 504a (e.g., a laptop computer), 504b (e.g., a smart phone), 504c (e.g., a personal computer) can each include the same characteristics, structure, components, attributes, and/or functionality as that of external computing device 504.

In some embodiments of the present disclosure, network(s) 506 can couple (e.g., communicatively) one or more of wearable devices 100a, 100b, 100c to server system 604 and/or one or more of external computing devices 504a, 504b, 504c. In some embodiments, one or more of external computing devices 504a, 504b, 504c and/or one or more of wearable devices 100a. 100b, 100c can be interconnected in a local area network (LAN) 602 or another type of communication interconnection that can be connected to (e.g., communicatively coupled to) network(s) 506. LAN 602 according to example embodiments can interconnect one or more of external computing devices 504a, 504b, 504c, as well as one or more of wearable devices 100a, 100b, 100c. In some embodiments, one or more of wearable devices 100a, 100b, 100c and/or one or more of external computing devices 504a, 504b, 504c can be connected to (e.g., communicatively coupled to) network(s) 506 and/or server system 604, indirectly, through LAN 602. In some embodiments, one or more of wearable devices 100a, 100b, 100c can be directly connected to (e.g., communicatively coupled to) network(s) 506 and/or indirectly connected to network(s) 506 through LAN 602. For instance, in the example embodiment depicted in FIG. 6, wearable device 100b can be connected to (e.g., communicatively coupled to) external computing device 504b (e.g., a smart phone) through, for example, a Bluetooth connection. In this embodiment, external computing device 504b can be connected to (e.g., communicatively coupled to) server system 604 through network(s) 506 and wearable device 100b can also be connected to (e.g., communicatively coupled to) server system 604 through network 506.

In the example embodiment depicted in FIG. 6, server system 604 can collect detected physiological and/or environmental sensor readings from one or more of wearable devices 100a, 100b, 100c. In some embodiments, server system 604 can also collect TBSS metric values of one or more users 10a, 10b, 10c from one or more of wearable devices 100a, 100b, 100c and/or from one or more of external computing devices 504a, 504b, 504c.

For example, in the embodiment depicted in FIG. 6, wearable device 100a is not associated with an external computing device, therefore it can transmit physiological data collected during a sleep session of user 10a to server system 604. In this embodiment, server system 604 can analyze the received data to calculate the TBSS metric of user 10a (e.g., by obtaining or generating the above-described plurality of sleep stages and using such sleep stages to calculate user's 10a TBSS metric in accordance with one or more embodiments described herein). In this embodiment, server system 604 can transmit an intelligent notification, user's 10a TBSS metric, and/or one or more sleep quality recommendations back to wearable device 100a.

As another example, in the embodiment depicted in FIG. 6, wearable device 100b can transmit physiological data collected during a sleep session of user 10b to server system 604 and external computing device 504a. In this embodiment, external computing device 504a can analyze the received data to calculate the TBSS metric of user 10b (e.g., by obtaining or generating the above-described plurality of sleep stages and using such sleep stages to calculate user's 10b TBSS metric in accordance with one or more embodiments described herein). In this embodiment, server system 604 can use the received physiological data of user 10b to update a user profile for user 10b that can be stored in a profiles database 612 (e.g., a log) that can be stored on a memory 608 that can be included in, coupled to, and/or otherwise associated with server system 604.

In some embodiments, server system 604 can be implemented on one or more standalone data processing apparatuses or a distributed network of computers. In some embodiments, server system 604 can employ various virtual devices and/or services of third-party service providers (e.g., third-party cloud service providers) to provide the underlying computing resources and/or infrastructure resources of server system 604. In some embodiments, server system 604 can include, but is not limited to, a handheld computer, a tablet computer, a laptop computer, a desktop computer, or a combination of any two or more of these data processing devices or other data processing devices.

Server system 604 according to example embodiments can include one or more processors or processing units 606 (denoted as “processor(s) 606” in FIG. 6) such as, for instance, one or more CPUs. In these or other embodiments, sever system 604 can include one or more network interfaces 614 that can include, for example, an input/output (I/O) interface to external computing device 504a, 504b, and/or 504c and/or wearable devices 100a, 100b, and/or 100c. In some embodiments, server system 604 can include memory 608, and one or more communication buses for interconnecting these components.

Memory 608 according to example embodiments can include high-speed random-access memory such as, for instance, DRAM, SRAM, DDR RAM, or other random-access solid-state memory devices; and, optionally, can include non-volatile memory such as, for example, one or more magnetic disk storage devices, one or more optical disk storage devices, one or more flash memory devices, or one or more other non-volatile solid state storage devices. Memory 608 according to example embodiments, optionally, can include one or more storage devices that can be remotely located from processor(s) or processing unit(s) 606. Memory 608 according to example embodiments, or alternatively the non-volatile memory within memory 608, can include a non-transitory computer readable storage medium. In some embodiments, memory 608, or the non-transitory computer readable storage medium of memory 608, can store one or more programs, modules, and data structures. In these embodiments, such programs, modules, and data structures can include, but not be limited to, one or more of an operating system that can include procedures for handling various basic system services and for performing hardware dependent tasks, a network communication module for connecting server system 604 to other computing devices (e.g., wearable device 100a, 100b, and/or 100c and/or external computing device 504a, 504b, and/504c) connected to network(s) 506 via network interface(s) 614 (e.g., wired or wireless).

Memory 608 according to example embodiments can include TBSS metric module 113 described above with reference to FIG. 4 that can use collected physiological and/or environmental data of one or more users 10a, 10b, 10c (e.g., received from one or more wearable devices 100a. 100b, 100c or one or more external computing devices 504a, 504b, 504c) to calculate TBSS metrics corresponding respectively to users 10a, 10b, 10c. In one embodiment, server system 604 can implement TBSS metric module 113 to calculate a TBSS metric for each of user 10a, 10b, 10c by obtaining the above-described plurality of sleep stages for each user 10a, 10b, 10c and using such sleep stages to calculate each TBSS metric in accordance with one or more embodiments described herein. In another embodiment, server system 604 can implement TBSS metric module 113 to calculate each TBSS metric for each user 10a, 10b, 10c by generating the above-described plurality of sleep stages for each user 10a, 10b, 10c and using such sleep stages to calculate each TBSS metric in accordance with one or more embodiments described herein. For instance, in this embodiment, server system 604 can use the above-described classifier(s) to generate the plurality of sleep stages for each user 10a, 10b, 10c and use such sleep stages to calculate each TBSS metric for each user 10a, 10b, 10c in accordance with one or more embodiments described herein. In this embodiment, server system 604 can implement the classifier(s) to generate the plurality of sleep stages for each user 10a, 10b, 10c using physiological data (e.g., heart rate, motion, temperature, respiration) of each user 10a, 10b, 10c that can be captured, collected, and/or measured by wearable devices 100a, 100b, 100c, respectively.

Memory 608 according to example embodiments can also include profiles database 612 that can store user profiles for users 10a, 10b, 10c. In some embodiments, a respective user profile for a user can include, for instance: a user identifier (e.g., an account name or handle); login credentials (e.g., login credentials to sleep quality management system 600); email address or preferred contact information; wearable device information (e.g., model number); demographic parameters for the user (e.g., age, gender, occupation); historical sleep quality information of the user; historical TBSS metrics of the user; and/or identified sleep quality trends of the user (e.g., particularly restless sleeper).

In some embodiments, collected physiological information of a plurality of users such as, for instance, users 10a, 10b, 10c of can provide for more robust population-normalized sleep metrics. For example, user 10a can be a 35 year old female veterinarian and user 10b can be a 34 year old female veterinarian, and each of their respective historical sleep quality physiological data and/or metrics can be used in the determination of one or more population-normalized sleep quality metrics for each other, due to their closely aligned demographic characteristics. In some embodiments, a user can opt in or opt out of providing sleep quality assessment information to a population-normalization determination for other users. In some embodiments, a user's sleep quality information can be incorporated into population-normalized sleep quality metric information used to determine that user's own values for one or more sleep quality metrics.

In at least one embodiment described herein, server system 604 can record, in profiles database 612, the TBSS metrics corresponding to users 10a, 10b, 10c. In this embodiment, server system 604 can compare a TBSS metric of a certain user 10a, 10b, or 10c with the TBSS metrics of other users and further classify such a certain user in a defined sleep pattern category (e.g., an insomnia sleep pattern category) based at least in part on such comparison of users' TBSS metrics. In some embodiments, to perform the comparison and/or classification operations described above, server system 604 can use one or more of the above-described classifiers and/or another classifier that can compare one or more TBSS metrics of a certain user with one or more TBSS metrics of one or more other users and classify such a certain user in a defined sleep pattern category based on such comparison.

In at least one embodiment of the present disclosure, server system 604 can identify a defined sleep pattern of a certain user 10a, 10b, or 10c based at least in part on a TBSS metric and/or historical TBSS metrics corresponding to such a certain user. For instance, in this or another embodiment, by comparing one or more TBSS metrics of such a certain user with one or more TBSS metrics of one or more other users and classifying such a certain user in a defined sleep pattern category based on such comparison as described above (e.g., via one or more classifiers), server system 604 can thereby determine that such a certain user's sleep pattern corresponds to a certain sleep pattern such as, for example, an insomnia sleep pattern. In some embodiments, based at least in part on (e.g., in response to) identifying such a defined sleep pattern of such a certain user, server system 604 can further determine a defined sleep condition diagnosis and/or a defined sleep condition prognosis that can be associated with sleep quality of such a certain user. For instance, in these or other embodiments, based at least in part on (e.g., in response to) determining such a certain user's sleep pattern corresponds to an insomnia sleep pattern as described above, server system 604 can further diagnose such a certain user as an insomniac.

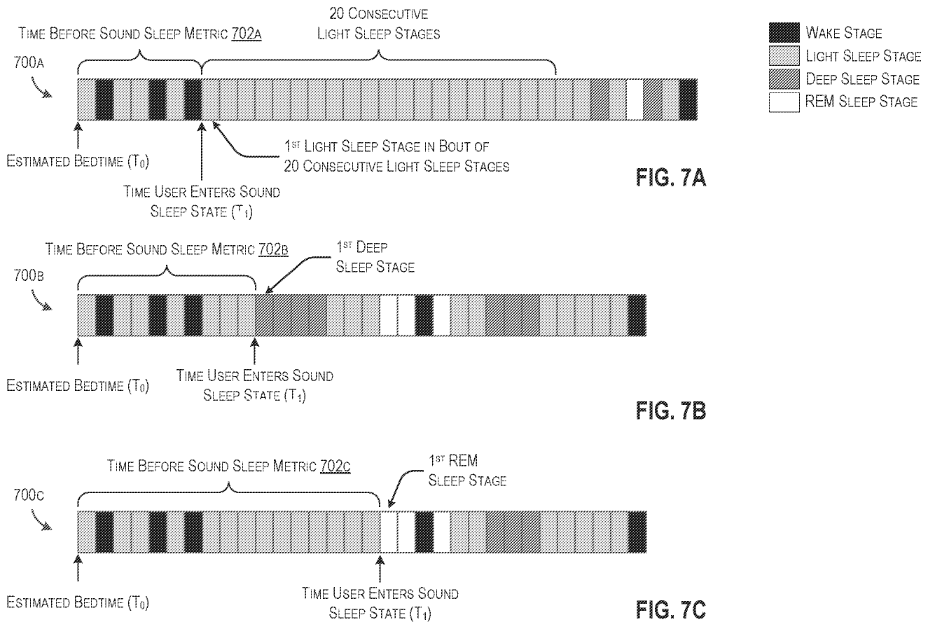

FIGS. 7A, 7B, and 7C each illustrate a diagram of example, non-limiting sleep stages 700a, 700b, 700c, respectively, according to one or more example embodiments of the present disclosure. Sleep stages 700a, 700b, 700c illustrated in the example embodiments depicted in FIGS. 7A, 7B, and 7C, respectively, can constitute and/or include a plurality of sleep stages as defined herein that can be associated with a sleep session of a user (e.g., a sleep session of user 10 that can last, for instance, one or more hours).

In the embodiments depicted in FIGS. 7A, 7B, and 7C, sleep stages 700a, 700b, 700c can each include, for example: a wake stage (represented by black boxes in FIGS. 7A, 7B, and 7C); a light sleep stage (represented by light gray boxes in FIGS. 7A, 7B, and 7C); a deep sleep stage (represented by dark gray boxes in FIGS. 7A, 7B, and 7C); and an REM sleep stage (represented by white boxes in FIGS. 7A, 7B, and 7C). In these embodiments, sleep stages 700a, 700b, 700c can each include, for example: a wake stage where the user is awake; a light sleep stage where the user is in a restful pre-sleep state and/or a relaxing state of stillness; a deep sleep stage where the user is in a true, continuous, and/or quality sleep state; and/or an REM sleep stage where the user experiences REM and is thus in a true, continuous, and/or quality sleep state.

In at least one embodiment of the present disclosure, one or more defined sleep stages of sleep stages 700a, 700b, 700c can be indicative of a defined sleep state of the user (e.g., the sound sleep state defined above). In this or another embodiment, such defined sleep stage(s) can constitute and/or include a defined quantity of one or more of the sleep stages in sleep stages 700a, 700b, 700c. For example, in one embodiment, the one or more defined sleep stages can constitute and/or include: a defined quantity (e.g., 10, 20, 30) of the light sleep stage; a defined quantity (e.g., 1, 2) of the deep sleep stage; and/or a defined quantity (e.g., 1, 2) of the REM sleep stage.

In some embodiments described herein, each sleep stage of sleep stages 700a, 700b, 700c and each defined sleep stage of the one or more defined sleep stages can be defined by a certain time interval (e.g., 30 seconds, 1 minute, 2 minutes) such that each sleep stage in each of sleep stages 700a, 700b, 700c are defined by the same duration of time (e.g., 30 seconds, 1 minute, 2 minutes). In some embodiments, each sleep stage of sleep stages 700a, 700b, 700c and each defined sleep stage of the one or more defined sleep stages can be defined by a certain time interval (e.g., 30 seconds, 1 minute, 2 minutes) that corresponds to a discrete time interval (e.g., 30 seconds, 1 minute, 2 minutes) in the user's sleep session.

In each of the example embodiments depicted in FIGS. 7A, 7B, and 7C, each sleep stage of sleep stages 700a, 700b, and 700c can be 1 minute in duration. That is, for instance, in these example embodiments, each wake stage, each light sleep stage, each deep sleep stage, and each REM sleep stage depicted in FIGS. 7A, 7B, and 7C can be 1 minute in duration. As such, in these example embodiments, each rectangle that represents a sleep stage in sleep stages 700a, 700b, and 700c can represent a 1-minute duration.

In one or more embodiments of the present disclosure, a time at which a certain light sleep stage (e.g., the 1^{st} light sleep stage) begins in the user's sleep session can correspond to the estimated bedtime of the user as defined above. In some embodiments, a time at which the above-described one or more defined sleep stages begin can correspond to a time at which the user enters the defined sleep state (e.g., the sound sleep state defined above).

In the example embodiment depicted in FIG. 7A, the one or more defined sleep stages described above can constitute and/or include a defined quantity of the light sleep stages such as, for instance, 20 light sleep stages (e.g., 20 consecutive light sleep stages). For instance, in the example embodiment depicted in FIG. 7A, the one or more defined sleep stages described above can constitute and/or include 20 consecutive, uninterrupted light sleep stages in the user's sleep session. As such, in this example embodiment, the one or more defined sleep stages can constitute and/or include a 20-minute, uninterrupted bout of light sleep. In this or another embodiment, a time (e.g., T₀ in FIG. 7A) at which the 1^{st} light sleep stage begins in the user's sleep session can correspond to the estimated bedtime of the user as defined above. In this or another embodiment, a time (e.g., T₁ in FIG. 7A) at which the 1^{st} light sleep stage begins in the bout of 20 consecutive, uninterrupted light sleep stages can correspond to the time at which the user enters the sound sleep state defined above. To calculate TBSS metric 702a illustrated in the example embodiment depicted in FIG. 7A, a computing device described herein (e.g., wearable device 100, 100a, 100b, and/or 100c, external computing device 504, 504a, 504b, and/or 504c, and/or server system 604) can subtract the time (e.g., T₀ in FIG. 7A) at which the 1^{st} light sleep stage begins in the user's sleep session from the time (e.g., T₁ in FIG. 7A) at which the 1^{st} light sleep stage begins in the bout of 20 consecutive, uninterrupted light sleep stages in the user's sleep session.

In the example embodiment depicted in FIG. 7B, the one or more defined sleep stages described above can constitute and/or include a defined quantity of the deep sleep stages such as, for instance, 1 deep sleep stage. In this or another embodiment, a time (e.g., To in FIG. 7B) at which the 1^{st} light sleep stage begins in the user's sleep session can correspond to the estimated bedtime of the user as defined above. In this or another embodiment, a time (e.g., T₁ in FIG. 7B) at which the deep sleep stage (e.g., the 1^{st} deep sleep stage) begins in the user's sleep session can correspond to the time at which the user enters the sound sleep state defined above. To calculate TBSS metric 702b illustrated in the example embodiment depicted in FIG. 7B, a computing device described herein (e.g., wearable device 100, 100a, 100b, and/or 100c, external computing device 504, 504a, 504b, and/or 504c, and/or server system 604) can subtract the time (e.g., To in FIG. 7B) at which the 1^{st} light sleep stage begins in the user's sleep session from the time (e.g., T₁ in FIG. 7B) at which the deep sleep stage (e.g., the 1^{st} deep sleep stage) begins in the user's sleep session.

In the example embodiment depicted in FIG. 7C, the one or more defined sleep stages described above can constitute and/or include a defined quantity of the REM sleep stages such as, for instance, 1 REM sleep stage. In this or another embodiment, a time (e.g., T₀ in FIG. 7C) at which the 1^{st} light sleep stage begins in the user's sleep session can correspond to the estimated bedtime of the user as defined above. In this or another embodiment, a time (e.g., T₁ in FIG. 7C) at which the REM sleep stage (e.g., the 1^{st} REM sleep stage) begins in the user's sleep session can correspond to the time at which the user enters the sound sleep state defined above. To calculate TBSS metric 702c illustrated in the example embodiment depicted in FIG. 7C, a computing device described herein (e.g., wearable device 100, 100a, 100b, and/or 100c, external computing device 504, 504a, 504b, and/or 504c, and/or server system 604) can subtract the time (e.g., To in FIG. 7C) at which the 1^{st} light sleep stage begins in the user's sleep session from the time (e.g., T₁ in FIG. 7C) at which the REM sleep stage (e.g., the 1^{st} REM sleep stage) begins in the user's sleep session.

### Example Methods

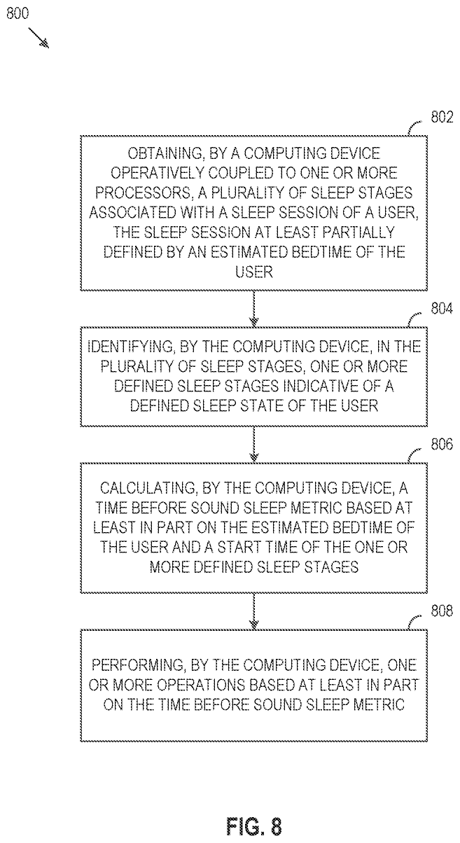

FIG. 8 illustrates a flow diagram of an example, non-limiting computer-implemented method 800 according to one or more example embodiments of the present disclosure. Computer-implemented method 800 can be implemented using, for instance, wearable device 100, 100a, 100b, 100c, 504, 504a, 504b, 504c, or 604 described above with reference to the example embodiments depicted in FIGS. 1, 2, 3, 4, 5, and 6.

The example embodiment illustrated in FIG. 8 depicts operations performed in a particular order for purposes of illustration and discussion. Those of ordinary skill in the art, using the disclosures provided herein, will understand that various operations or steps of computer-implemented method 800 or any of the other methods disclosed herein can be adapted, modified, rearranged, performed simultaneously, include operations not illustrated, and/or modified in various ways without deviating from the scope of the present disclosure.

At 802, computer-implemented method 800 can include obtaining (e.g., via network(s) 506, LAN 602), by a computing device (e.g., wearable device 100, 100a, 100b, and/or 100c, external computing device 504, 504a, 504b, and/or 504c, and/or server system 604) operatively coupled to one or more processors (e.g., processor(s) 181, processor(s) 606), a plurality of sleep stages (e.g., sleep stages 700a, 700b, or 700c) associated with a sleep session of a user (e.g., user 10), the sleep session at least partially defined by an estimated bedtime (e.g., To in FIG. 7A, 7B, or 7C) of the user.

At 804, computer-implemented method 800 can include identifying, by the computing device, in the plurality of sleep stages, one or more defined sleep stages (e.g., a defined quantity (e.g., 10, 20, 30) of the light sleep stage; a defined quantity (e.g., 1, 2) of the deep sleep stage; and/or a defined quantity (e.g., 1, 2) of the REM sleep stage) indicative of a defined sleep state (e.g., the sound sleep state defined above) of the user.

At 806, computer-implemented method 800 can include calculating, by the computing device, a time before sound sleep metric (e.g., the TBSS metric defined herein) based at least in part on the estimated bedtime (e.g., To in FIG. 7A, 7B, or 7C) of the user and a start time (e.g., T₁ in FIG. 7A, 7B, or 7C) of the one or more defined sleep stages.

At 810, computer-implemented method 800 can include performing, by the computing device, one or more operations (e.g., generating and/or providing an intelligent notification, the TBSS metric, and/or one or more sleep quality recommendations to the user and/or another computing device, implementing one or more sleep promoting features of the computing device, another computing device, and/or a smart system defined above; and/or identifying a defined sleep pattern of the user and determining a defined sleep condition diagnosis or a defined sleep condition prognosis) based at least in part on the time before sound sleep metric.

### Additional Disclosure

The technology discussed herein makes reference to servers, databases, software applications, and other computer-based systems, as well as actions performed by, and information sent to and from such systems. The inherent flexibility of computer-based systems allows for a great variety of possible configurations, combinations, and divisions of tasks and functionality between and among components. For instance, processes discussed herein can be implemented using a single device or component or multiple devices or components working in combination. Databases and applications can be implemented on a single system or distributed across multiple systems. Distributed components can operate sequentially or in parallel.

While the present subject matter has been described in detail with respect to various specific example embodiments thereof, each example is provided by way of explanation, not limitation of the disclosure. Those skilled in the art, upon attaining an understanding of the foregoing, can readily produce alterations to, variations of, and equivalents to such embodiments. Accordingly, the subject disclosure does not preclude inclusion of such modifications, variations and/or additions to the present subject matter as would be readily apparent to one of ordinary skill in the art. For instance, features illustrated or described as part of one embodiment can be used with another embodiment to yield a still further embodiment. Thus, it is intended that the present disclosure covers such alterations, variations, and equivalents.

What is claimed is:

1. A computing device, comprising:
one or more processors: and one or more computer-readable media that store instructions that, when executed by the one or more processors, cause the computing device to perform operations, the operations comprising:
obtaining a plurality of sleep stages associated with a sleep session of a user, the sleep session at least partially defined by an estimated bedtime of the user;
identifying, in the plurality of sleep stages, one or more defined sleep stages indicative of a defined sleep state of the user;
calculating a time before sound sleep metric based at least in part on the estimated bedtime of the user and a start time of the one or more defined sleep stages; and
performing one or more operations based at least in part on the time before sound sleep metric.

2. The computing device of claim 1, wherein the time before sound sleep metric is indicative of a time period between the estimated bedtime of the user and a time when the user enters the defined sleep state.

3. The computing device of claim 1, wherein the plurality of sleep stages comprises at least one of a wake stage, a light sleep stage, a deep sleep stage, or a rapid eye movement sleep stage, and wherein the one or more defined sleep stages comprise a defined quantity of at least one of the light sleep stage, the deep sleep stage, or the rapid eye movement sleep stage.

4. The computing device of claim 1, wherein calculating the time before sound sleep metric based at least in part on the estimated bedtime of the user and the start time of the one or more defined sleep stages comprises:
calculating a difference in time between the estimated bedtime of the user and the start time of the one or more defined sleep stages.

5. The computing device of claim 1, wherein performing the one or more operations based at least in part on the time before sound sleep metric comprises:
generating an intelligent notification comprising the time before sound sleep metric; and
providing the intelligent notification to at least one of the user or a second computing device.

6. The computing device of claim 1, wherein performing the one or more operations based at least in part on the time before sound sleep metric comprises:
generating one or more sleep quality recommendations based at least in part on the time before sound sleep metric; and
providing an intelligent notification comprising at least one of the time before sound sleep metric or the one or more sleep quality recommendations to at least one of the user or a second computing device.

7. The computing device of claim 1, wherein performing the one or more operations based at least in part on the time before sound sleep metric comprises:
implementing one or more sleep promoting features of at least one of the computing device or a second computing device based at least in part on the time before sound sleep metric.

8. The computing device of claim 1, wherein performing the one or more operations based at least in part on the time before sound sleep metric comprises:
recording, in a database, at least one of the time before sound sleep metric or one or more additional time before sound sleep metrics corresponding to the user, the one or more additional time before sound sleep metrics being calculated based at least in part on at least one additional plurality of sleep stages associated with one or more additional sleep sessions of the user.

9. The computing device of claim 8, wherein performing the one or more operations based at least in part on the time before sound sleep metric comprises:
comparing at least one of the time before sound sleep metric or the one or more additional time before sound sleep metrics to one or more second time before sound sleep metrics corresponding respectively to one or more second users; and
classifying the user in a defined sleep pattern category based at least in part on comparison of at least one of the time before sound sleep metric or the one or more additional time before sound sleep metrics to the one or more second time before sound sleep metrics.

10. The computing device of claim 1, wherein performing the one or more operations based at least in part on the time before sound sleep metric comprises:
identifying a defined sleep pattern of the user based at least in part on at least one of the time before sound sleep metric or one or more additional time before sound sleep metrics corresponding to the user; and
determining at least one of a defined sleep condition diagnosis or a defined sleep condition prognosis associated with sleep quality of the user based at least in part on the defined sleep pattern.

11. A computer-implemented method of assessing sleep quality and facilitating sleep quality alteration, the computer-implemented method comprising:
obtaining, by a computing device operatively coupled to one or more processors, a plurality of sleep stages associated with a sleep session of a user, the sleep session at least partially defined by an estimated bedtime of the user;
identifying, by the computing device, in the plurality of sleep stages, one or more defined sleep stages indicative of a defined sleep state of the user;
calculating, by the computing device, a time before sound sleep metric based at least in part on the estimated bedtime of the user and a start time of the one or more defined sleep stages; and
performing, by the computing device, one or more operations based at least in part on the time before sound sleep metric.

12. The computer-implemented method of claim 11, wherein the time before sound sleep metric is indicative of a time period between the estimated bedtime of the user and a time when the user enters the defined sleep state.

13. The computer-implemented method of claim 11, wherein the plurality of sleep stages comprises at least one of a wake stage, a light sleep stage, a deep sleep stage, or a rapid eye movement sleep stage, and wherein the one or more defined sleep stages comprise a defined quantity of at least one of the light sleep stage, the deep sleep stage, or the rapid eye movement sleep stage.

14. The computer-implemented method of claim 11, wherein calculating, by the computing device, the time before sound sleep metric based at least in part on the estimated bedtime of the user and the start time of the one or more defined sleep stages comprises:
calculating, by the computing device, a difference in time between the estimated bedtime of the user and the start time of the one or more defined sleep stages.

15. The computer-implemented method of claim 11, wherein performing, by the computing device, the one or more operations based at least in part on the time before sound sleep metric comprises:
generating, by the computing device, an intelligent notification comprising the time before sound sleep metric; and
providing, by the computing device, the intelligent notification to at least one of the user or a second computing device.

16. The computer-implemented method of claim 11, wherein performing, by the computing device, the one or more operations based at least in part on the time before sound sleep metric comprises:
generating, by the computing device, one or more sleep quality recommendations based at least in part on the time before sound sleep metric; and
providing, by the computing device, an intelligent notification comprising at least one of the time before sound sleep metric or the one or more sleep quality recommendations to at least one of the user or a second computing device.

17. The computer-implemented method of claim 11, wherein performing, by the computing device, the one or more operations based at least in part on the time before sound sleep metric comprises:
implementing, by the computing device, one or more sleep promoting features of at least one of the computing device or a second computing device based at least in part on the time before sound sleep metric.

18. The computer-implemented method of claim 11, wherein performing, by the computing device, the one or more operations based at least in part on the time before sound sleep metric comprises:
recording, by the computing device, in a database, at least one of the time before sound sleep metric or one or more additional time before sound sleep metrics corresponding to the user, the one or more additional time before sound sleep metrics being calculated based at least in part on at least one additional plurality of sleep stages associated with one or more additional sleep sessions of the user;
comparing, by the computing device, at least one of the time before sound sleep metric or the one or more additional time before sound sleep metrics to one or more second time before sound sleep metrics corresponding respectively to one or more second users; and
classifying, by the computing device, the user in a defined sleep pattern category based at least in part on comparison of at least one of the time before sound sleep metric or the one or more additional time before sound sleep metrics to the one or more second time before sound sleep metrics.

19. The computer-implemented method of claim 11, wherein performing, by the computing device, the one or more operations based at least in part on the time before sound sleep metric comprises:
identifying, by the computing device, a defined sleep pattern of the user based at least in part on the time before sound sleep metric; and
determining, by the computing device, at least one of a defined sleep condition diagnosis or a defined sleep condition prognosis associated with sleep quality of the user based at least in part on the defined sleep pattern.

20. One or more computer-readable media that store instructions that, when executed by one or more processors of a computing device, cause the computing device to perform operations, the operations comprising:
obtaining a plurality of sleep stages associated with a sleep session of a user, the sleep session at least partially defined by an estimated bedtime of the user;
identifying, in the plurality of sleep stages, one or more defined sleep stages indicative of a defined sleep state of the user, calculating a time before sound sleep metric based at least in part on the estimated bedtime of the user and a start time of the one or more defined sleep stages; and
performing one or more operations based at least in part on the time before sound sleep metric.
